(19) United States
(12) Patent Application Publication
(10) Pub. No.: US 2018/0236004 A1
(43) Pub. Date: Aug. 23, 2018

(54) STEM CELL-DERIVED OLIGODENDROCYTE PROGENITOR CELLS FOR THE TREATMENT OF WHITE MATTER STROKE

(71) Applicants: Asterias Biotherapeutics, Inc., Fremont, CA (US); The Regents of The University of California, Oakland, CA (US)

(72) Inventors: Edward D. Wirth, III, Mountain View, CA (US); Stanley T. Carmichael, Sherman Oaks, CA (US); Irene Lorenzo Llorente, Los Angeles, CA (US); Nathan C. Manley, San Jose, CA (US)

(73) Assignees: Asterias Biotherapeutics, Inc., Fremont, CA (US); The Regents of The University of California, Oakland, CA (US)

(21) Appl. No.: 15/753,135

(22) PCT Filed: Aug. 15, 2016
(86) PCT No.: PCT/US16/47109

§ 371 Date: Feb. 15, 2018

Related U.S. Application Data
(60) Provisional application No. 62/205,723, filed on Aug. 15, 2015.

(51) Int. Cl. A61K 35/30; A61K 47/42; A61K 9/00; C12N 5/079; C12N 5/073; A61P 43/00

(52) U.S. Cl. CPC A61K 35/30; A61K 47/42; A61K 9/0024; A61K 2035/124; C12N 5/0605; A61P 43/00; C12N 5/0622

(57) ABSTRACT

In various embodiments methods and compositions for improving a recovery of a subject after a cerebral ischemic injury, such as white matter stroke are provided. In various embodiments, the methods involve administering stem cell-derived oligodendrocyte progenitor cells into or directly adjacent to the infarct core in the brain of the subject.

### PRIORITY

This application claims priority to U.S. Provisional Application No. 62/205,723, filed on Aug. 15, 2015, the contents of which are hereby incorporated in their entirety.

### FIELD

In various embodiments, in vitro differentiated oligodendrocyte progenitor cells and methods of using the same in the treatment of cerebral ischemic injury are provided.

### BACKGROUND

Subcortical white matter stroke (WMS) constitutes up to 25% of the 795,000 new strokes occurring annually in the U.S. and is the second leading cause of dementia. During the normal human aging process, the white matter regions of the brain suffer progressive damage related to both overt and clinically silent ischemia. This type of ischemia is often termed “small vessel disease” as it occurs without occlusion of large cerebral arteries, and can occur without the clinical symptoms typical of stroke damage (Gorelick et al., (2011) Stroke 42(9):2672). Cerebral white matter lesions indicative of WMS are detected on brain imaging in asymptomatic individuals who have not suffered a stroke (Debette and Markus, (2010) British Medical Journal 341:c3666), and accumulate with age such that they are present in virtually all individuals over the age of 80 (de Leeuw et al., (2001) J Neurol Neurosurg Psychiatry 70:9). The degree of white matter injury closely correlates with abnormalities in cognition, balance and gait and carries an increased risk of death (Zheng et al., (2011) Stroke 42(7):2086; Debette and Markus, (2010) British Medical Journal 341:c3666). This progressive accumulation of ischemic white matter injury is the second leading cause of dementia and interacts with Alzheimer's disease to worsen and possibly accelerate this illness (Gorelick et al., (2011) Stroke 42(9):2672; DeCarli et al., (2013) J Alzheimers Dis. 33(Suppl 1):5417). Currently, there are no therapies available for WMS.

One potential viable therapy to restore neurological function in patients with WMS is cell transplantation. WMS has a very different pattern of cellular injury that occurs in traditional stroke models. The neural elements damaged in WMS include oligodendrocytes, oligodendrocyte progenitor cells (OPCs), astrocytes and axons. Human embryonic stem cell-derived oligodendrocyte cells (hESC-OPCs) provide an attractive candidate for WMS therapy. These cells are in a lineage that matches the most damaged cellular element in white matter ischemia—the cells of the oligodendrocyte lineage—and give rise to oligodendrocytes, the myelinating cells of the brain's white matter regions (Richardson et al., (2011) Neuron 70(4):661). Furthermore, cerebral white matter lesions are confluent and extensive in WMS, so that any candidate cell therapy would need to migrate to the site(s) of injury from the point of injection. OPCs have been shown to migrate extensively in CNS injury models (Goldman et al., (2012) Science 338(6106):491; Sun et al., (2013) PLoS One 8(2); e57534). Finally, OPCs are a source of secreted neurotrophic factors, such as BDNF, which may provide a direct reparative action on the ischemic white matter.

AST-OPC1 is a population of oligodendrocyte progenitor cells (OPCs) that are produced from human embryonic stem cells (hESCs) using a specific differentiation protocol (Nistor et al., (2005) Glia 49(3):385.) AST-OPC1 has been characterized by the expression of several molecules that are associated with oligodendrocyte precursors including Nestin, PDGRFα and NG2. The cells are further characterized by their minimal or lack of expression of markers known to be present in other cell types, such as neurons, astrocytes, endoderm, mesoderm, and hESCs. In vitro, AST-OPC1 also produces diffusible factors that support neurite extension from sensory neurons (Zhang et al., (2006) Stem Cells Dev. 15(6):943). AST-OPC1 has been shown to migrate extensively in spinal cord injury models (Keirstead et al., (2005) J Neurosci 25(19): 4694.

There is a need for developing novel therapies for cerebral ischemic injuries, including WMS. Moreover, there is a need for highly pure, clinical grade oligodendrocyte progenitor cells for use in cellular treatment for conditions requiring myelin repair and/or remyelination, such as WMS. The compositions and methods described herein meet these needs as well as other needs in the field.

### SUMMARY

In various embodiments described herein, methods are provided, inter alia, for treating a subject after a cerebral ischemic injury. In some embodiments the cerebral ischemic injury is white matter subcortical stroke. In certain embodiments, the subject is a human. In various embodiments the methods generally involve administering a therapeutically effective amount of pluripotent stem cell-derived oligodendrocyte progenitor cells into the brain of the subject. In some embodiments, the oligodendrocyte progenitor cells are derived from human embryonic stem cells. In other embodiments, the oligodendrocyte progenitor cells are derived from sources other than human embryonic stem cells, such as adult stem cells, induced pluripotent stem cells, and the like. In some embodiments, the methods comprise administering the pluripotent stem-cell derived oligodendrocyte progenitor cells into the infarct core. In other embodiments, the methods comprise administering the pluripotent stem-cell derived oligodendrocyte progenitor cells directly adjacent to the infarct core.

In certain embodiments, the pluripotent stem cell-derived oligodendrocyte progenitor cells are administered during the subacute time period after the ischemic injury. In some embodiments, the pluripotent stem cell-derived oligodendrocyte progenitor cells are administered during the early subacute time period after the ischemic injury. In other embodiments, the pluripotent stem cell-derived oligodendrocyte progenitor cells are administered during the late subacute time period.

In certain embodiments, the pluripotent stem cell-derived oligodendrocyte progenitor cells are administered using a depot delivery system. In some embodiments, the depot delivery system comprises a hydrogel. In some embodiments, the hydrogel comprises hyaluronate. In other embodiments, the hydrogel comprises thiolated hyaluronate. In some embodiments, the hydrogel comprises gelatin. In yet other embodiments, the hydrogel comprises thiolated gelatin. In some embodiments, the hydrogel comprises hyaluronate and gelatin. In other embodiments the hydrogel comprises thiolated hyaluronate and thiolated gelatin. In some embodiments, the hydrogel comprises a crosslinking agent. In other embodiments the hydrogel comprises thiolated hyaluronate, thiolated gelatin and a crosslinking agent.

In certain embodiments, methods for improving motor and/or cognitive function and/or speech of a subject after a cerebral ischemic injury are provided where the methods comprise administering a therapeutically effective amount of pluripotent stem cell-derived oligodendrocyte progenitor cells into or directly adjacent to the infarct core in the brain of said subject. In certain embodiments, the pluripotent stem cell-derived oligodendrocyte progenitor cells are human embryonic stem-cell derived oligodendrocyte progenitor cells. In other embodiments, the oligodendrocyte progenitor cells are derived from sources other than human embryonic stem cells, such as adult stem cells, induced pluripotent stem cells, and the like. In some embodiments the cerebral ischemic injury is white matter subcortical stroke. In certain embodiments, the subject is a human. In some embodiments, the method comprises administering the pluripotent stem cell-derived oligodendrocyte progenitor cells into the infarct core. In other embodiments, the method comprises administering the pluripotent stem cell-derived oligodendrocyte progenitor cells directly adjacent to the infarct core.

In certain embodiments, the present disclosure provides a container comprising a pharmaceutical composition for the treatment of white matter stroke, wherein the pharmaceutical composition comprises pluripotent stem cell-derived oligodendrocyte progenitor cells. In certain embodiments, the pluripotent stem cell-derived oligodendrocyte progenitor cells are human embryonic stem-cell derived oligodendrocyte progenitor cells. In other embodiments, the oligodendrocyte progenitor cells are derived from sources other than human embryonic stem cells, such as adult stem cells, induced pluripotent stem cells, and the like.

In yet other embodiments, the present disclosure provides a pharmaceutical composition for the treatment of subcortical while matter stroke, comprising pluripotent stem cell-derived oligodendrocyte progenitor cells. In certain embodiments, the pluripotent stem cell-derived oligodendrocyte progenitor cells are human embryonic stem-cell derived oligodendrocyte progenitor cells. In other embodiments, the oligodendrocyte progenitor cells are derived from sources other than human embryonic stem cells, such as adult stem cells, induced pluripotent stem cells, and the like. In certain embodiments, the pharmaceutical composition further comprises a depot delivery system. In some embodiments, the depot delivery system comprises a hydrogel. In some embodiments, the hydrogel comprises hyaluroate. In other embodiments, the hydrogel comprises thiolated hyaluronate. In some embodiments, the hydrogel comprises gelatin. In yet other embodiments, the hydrogel comprises thiolated gelatin. In some embodiments, the hydrogel comprises hyaluronate and gelatin. In other embodiments the hydrogel comprises thiolated hyaluronate and thiolated gelatin. In some embodiments, the hydrogel comprises a crosslinking agent. In other embodiments the hydrogel comprises thiolated hyaluronate, thiolated gelatin and a crosslinking agent.

### DESCRIPTION OF DRAWINGS

FIG. 1 is a representative magnetic resonance image of human white matter stroke taken 2 days after left hemispheric subcortical stroke. Arrow denotes white matter hyperintensity that was new in comparison to previous scans from the same patient. (Originally published in Sozmen et al., (2009) J. Neurosci Methods 180(2):261).

FIG. 2 shows an experimental timeline of Study 1 testing AST-OPC1 in a mouse model of white matter stroke. Key time points in the experimental design are shown, including AST-OPC1 transplantation seven days post-stroke and tissue processing fifteen days post-cell transplant. AST-OPC1 dose=100,000 cells/mouse as a single 1 μL injection. Abbreviations: L-NIO=N5-(1-iminoethyl)-L-ornithine, dihydrochloride.

FIG. 3 is a diagram of coronal mouse brain section indicating injection sites of L-NIO. Blue arrows indicated the three injection sites of L-NIO, delivered at an angle of 36° directly into the corpus callosum of each mouse brain to induce a focal ischemic lesion. Abbreviations: Cx=cortex; Str=striatum; WM=white matter.

FIGS. 4A-4B show representative fluorescent photomicrographs of astrocyte activation and axonal loss in the stroke-injured mouse brain. Image panels show fluorescent immunostaining of astrocytes (GFAP) and axons (NF200) three weeks after stroke injury in the uninjured contralateral hemisphere (FIG. 4A, control) and stroke-injured hemisphere (FIG. 4B, white matter stroke). In FIG. 4A, the left column depicts the merged fluorescent image for GFAP (green) and NF200 (red), the middle column depicts GFAP alone, and the right column depicts NF200 alone. In FIG. 4B, the left column depicts NF200 alone; the middle column depicts GFAP alone, and the right column depicts the merged fluorescent image for GFAP (green) and NF200 (red). The rows show increasing levels of magnification for the lesioned area (FIG. 4B) and contralateral side (FIG. 4A). Top rows=40×, middle rows=200×, bottom rows=600×. White boxes indicate the regions magnified in lower panels. The white dotted lines in FIG. 4A indicate the approximate borders of the corpus callosum. The asterisks indicate the lateral ventricle. Abbreviations: Cx=cortex; GFAP=glial fibrillary acid protein; NF200=neurofilament 200; Str=striatum; WM=white matter.

FIGS. 5A-5E shows representative fluorescent photomicrographs of activated microglia/immune cells in the stroke-injured mouse brain. FIG. 5A is a diagram of coronal mouse brain section indicating the regions shown in FIGS. 5B-5D. Relative microglial/immune cell activation three weeks after stroke injury as determined by Iba1 labeling (purple) is shown in the uninjured contralateral corpus callosum (FIG. 5B), and the injured ipsilateral corpus callosum (FIGS. 5C-5E). FIG. 5D and FIG. 5E show higher magnification images of the regions indicated in FIG. 5C. Dotted white line in FIG. 5C indicates the approximate border between infarct core and peri-infarct tissue. Magnification: FIG. 5B and FIG. 5C=600×; FIG. 5D and FIG. 5E=1000×. Abbreviations: Iba1=ionized calcium-binding adapter molecule 1.

FIGS. 6A-6D show representative fluorescent photomicrographs of myelin loss and oligodendrocyte response in the stroke-injured mouse brain. Relative myelin loss (MBP, green) and oligodendrocyte presence (OLIG2, red) three weeks after stroke injury in the uninjured contralateral corpus callosum (FIG. 6A, FIG. 6B), and the injured ipsilateral corpus callosum (FIG. 6C and FIG. 6 D). For FIG. 6A and FIG. 6C, left panel shows merged image for MBP and OLIG2, middle panel shows MBP alone, and right panel shows OLIG2 alone. FIG. 6B and FIG. 6D show higher magnification images of the merged images in FIG. 6A and FIG. 6C. FIG. 6E is a diagram of coronal mouse brain section showing regions depicted in FIGS. 6A-6D. Magnifications: FIG. 6A and FIG. 6C=600×; FIG. 6B and FIG. 6D=1000×. Abbreviations: MBP=myelin basic protein.

FIGS. 7A-7H show representative fluorescent photomicrographs of endogenous neurogenesis in the stroke-injured mouse brain. Relative neurogenesis (DCX, white), myelin presence (MBP, green), oligodendrocyte presence (OLIG2, red), and microglial/immune cell activation (IBA-1, blue) are shown for the lesioned corpus callosum three weeks after stroke injury. Top panels: FIG. 7A is a merged image of top panels, FIG. 7B is DCX alone, FIG. 7C is MBP alone, FIG. 7D is OLIG2 alone. Bottom panels: FIG. 7E is a merged image of bottom panels, FIG. 7F is IBA-1 alone, FIG. 7G is DCX alone, and FIG. 7H is OLIG2 alone. Red box in FIG. 7A depicts region magnified in FIG. 7E. Blue oval in FIG. 7B indicates a DCX positive cell. Magnifications: FIGS. 7A-7D=200×; FIGS. 7E-7H=600×. Abbreviations: DCX=doublecortin, IBA-1=ionized calcium-binding adapter molecule 1, MBP=myelin basic protein.

FIG. 8 is a diagram of coronal mouse brain section indicating cell injection site into the uninjured corpus callosum. Arrow indicates approximate cell injection site within the uninjured corpus callosum. Abbreviations: Cx=cortex; Str=striatum; WM=white matter.

FIGS. 9A-9D show representative fluorescent photomicrographs of astrocyte and microglial/immune cell activation in the uninjured mouse brain two weeks after AST-OPC1 transplantation. Image panels show fluorescent immunostaining of astrocytes (GFAP) and activated microglia/immune cells (Iba1/IBA-1) within the contralateral (FIG. 9A, FIG. 9C) and ipsilateral (FIG. 9B, FIG. 9D) two weeks after AST-OPC1 transplantation into the uninjured corpus callosum. In FIG. 9A and FIG. 9C, the left column depicts the merged fluorescent image for GFAP (green) and Iba1 (red), the middle column depicts GFAP alone, and the right column depicts Iba1 alone. In FIG. 9B and FIG. 9D, the left column depicts Iba1 alone; the middle column depicts GFAP alone, and the right column depicts the merged fluorescent image for GFAP (green) and Iba1 (red). The white boxes in FIG. 9A and FIG. 9B indicate the magnified regions shown in FIG. 9C and FIG. 9D. Magnifications: FIG. 9A and FIG. 9B=40×; FIG. 9C and FIG. 9D=600×. Abbreviations: Cx=cortex; GFAP=glial fibrillary acid protein; Iba1/IBA-1=ionized calcium-binding adapter molecule 1; Str=striatum.

FIGS. 10A-10E show representative fluorescent photomicrographs indicating no myelin loss and minimal immature neuronal migration in the uninjured mouse brain two weeks after AST-OPC1 transplantation. Image panels show fluorescent immunostaining of myelin (MBP) and immature neurons (DCX) within the contralateral (FIG. 10A, FIG. 10C) and ipsilateral (FIG. 10B, FIG. 10D) two weeks after AST-OPC1 transplantation into the uninjured corpus callosum. In FIG. 10A and FIG. 10B, the left column depicts the merged fluorescent image for MBP (green) and DCX (red), the middle column depicts MBP alone, and the right column depicts DCX alone. In FIG. 10C and FIG. 10D, MBP staining alone is shown in a magnified subregion. FIG. 10E is a diagram of mouse coronal brain section indicating regions shown in fluorescent micrographs. Magnifications: FIG. 10A and FIG. 10B=40×; FIG. 10C and FIG. 10D=600×. Abbreviations: DCX=doublecortin; MBP=myelin basic protein.

FIGS. 11A-11D show representative fluorescent photomicrographs indicating increased oligodendrocyte response in the uninjured mouse brain two weeks after AST-OPC1 transplantation. Image panels show fluorescent immunostaining of oligodendrocytes (OLIG2), myelin (MBP), and activated microglia/immune cells (IBA-1) within the contralateral (FIGS. 11A-11B) and ipsilateral (FIGS. 11C-11D) two weeks after AST-OPC1 transplantation into the uninjured corpus callosum. In FIG. 11A, panel 1 (left-most) depicts the merged fluorescent image for MBP (green) and OLIG2 (red), and IBA-1 (blue), panel 2 depicts IBA-1 alone, panel 3 depicts MBP alone, and panel 4 depicts OLIG2 alone. In FIG. 11B, the merged image for IBA-1 and OLIG2 from FIG. 11A is shown. In FIG. 11C, panel 1 (left-most) depicts OLIG2 alone, panel 2 depicts MBP alone, panel 3 depicts IBA-1 alone, and panel 4 depicts the merged fluorescent image for MBP (green) and OLIG2 (red), and IBA-1 (blue). In FIG. 11D, the merged image for IBA-1 and OLIG2 from FIG. 11C is shown. The diagram between FIG. 11A and FIG. 11C indicates the regions shown in the fluorescent micrographs. Magnification=200×. Abbreviations: Iba1/IBA-1=ionized calcium-binding adapter molecule; MBP=myelin basic protein.

FIG. 12 is a diagram of coronal mouse brain section indicating cell injection site into the stroke-lesioned corpus callosum. Blue arrows indicate the approximate injection sites for L-NIO to induce the stroke lesion. Black arrow indicates the approximate location of the cell injection site within the stroke lesion core (red area). The approximate lesion core is shown in blue. Abbreviations: Cx=cortex; Str=striatum; WM=white matter.

FIGS. 13A-13F show representative fluorescent photomicrographs of astrocyte and microglia/immune cell activation in the mouse brain following stroke injury and AST-OPC1 transplantation inside the lesion core. Image panels show fluorescent immunostaining of astrocytes (GFAP) and activated microglia/immune cells (IBA-1) in the uninjured contralateral hemisphere (FIGS. 13A-13C) and stroke-injured/AST-OPC1 transplanted hemisphere (FIGS. 13D-13F) three weeks post-stroke and two weeks post-AST-OPC1 transplantation. FIG. 13A, FIG. 13B, FIG. 13D, and FIG. 13E shown GFAP immunostaining at low magnification (FIG. 13A, FIG. 13D) and high magnification (FIG. 13B, FIG. 13E). FIG. 13C and FIG. 13F show at high magnification, the merged image of GFAP (green) and IBA-1 (orange) in the left panel, GFAP alone in the middle panel, and IBA-1 alone in the right panel. Asterisk indicates the approximate lesion core. Magnifications: FIG. 13A and FIG. 13D=40×; FIG. 13B and FIG. 13E=200×; FIG. 13C and FIG. 13F=600×. Abbreviations: Cx=cortex; GFAP=glial fibrillary acid protein; IBA-1=ionized calcium-binding adapter molecule; Str=striatum.

FIGS. 14A-14D show representative fluorescent photomicrographs of astrocyte activation and axonal loss in the mouse brain following stroke injury and AST-OPC1 transplantation inside the lesion core. Image panels show fluorescent immunostaining of astrocytes (GFAP) and axons (NF200) in the stroke-injured/AST-OPC1 transplanted hemisphere three weeks post-stroke and two weeks post-AST-OPC1 transplantation. FIG. 14A shows the merged image for GFAP (red) and NF200 (blue) within the stroke lesion. FIG. 14B shows NF200 staining alone, and FIG. 14C shows GFAP staining alone. The white dotted lines in FIGS. 14A-14C indicate the approximate border of the corpus callosum. The asterisk in FIGS. 14A-14C indicate the approximate core of the stroke lesion. FIG. 14D is a diagram of coronal mouse brain section indicating regions shown in FIGS. 14A-14C. Magnification=200×. Abbreviations: Cx=cortex; GFAP=glial fibrillary acid protein; Str=striatum.

FIGS. 15A-15D show representative fluorescent photomicrographs of AST-OPC1 location, myelin loss, and endogenous neurogenesis in the mouse brain following stroke injury and AST-OPC1 transplantation inside the lesion core. Image panels show fluorescent immunostaining of AST-OPC1 (Hnuc) and myelin (MBP) and immature neurons (DCX) in the stroke-injured/AST-OPC1 transplanted hemisphere three weeks post-stroke and two weeks post-AST-OPC1 transplantation. In FIGS. 15A-15C panel 1 (left-most) shows the merged image for Hnuc (blue), MBP (green), and DCX (red) within the stroke lesion. FIG. 15D is a diagram of coronal mouse brain section indicating regions shown in FIGS. 15A-15C. Magnifications: FIG. 15A=200×, FIG. 15B=1200×, FIG. 15C=2000×. Abbreviations: DCX=doublecortin; Hnuc=anti-human nuclei; MBP=myelin basic protein.

FIGS. 16A-16D show representative fluorescent photomicrographs of microglia/immune cell activation, myelin loss, and oligodendrocyte response in the mouse brain following stroke injury and AST-OPC1 transplantation inside the lesion core. Image panels show fluorescent immunostaining of activated microglia/immune cells (Iba1/IBA-1), myelin (MBP), and oligodendrocytes (OLIG2) in the stroke-injured/AST-OPC1 transplanted hemisphere (FIG. 16A, FIG. 16C) and the uninjured contralateral hemisphere (FIG. 16B, FIG. 16D) three weeks post-stroke and two weeks post-AST-OPC1 transplantation. In FIG. 16A, panel 1 (left-most) shows the merged image for IBA-1 (blue), MBP (green), and OLIG2 (red) within the stroke lesion. Panel 2 shows IBA-1 alone, panel 3 shows MBP alone, and panel 4 shows OLIG2 alone. In FIG. 16B, panel 1 (left-most) shows OLIG2 alone, panel 2 shows MBP alone, panel 3 shows IBA-1 alone, and panel 4 shows the merged image of all three stains. FIG. 16C and FIG. 16D show the merged images for IBA-1 (blue) and OLIG2 (red) only. The diagram between FIG. 16A and FIG. 16B indicates the regions shown in the fluorescent micrographs. Magnification=200×. Abbreviations: IBA-1=ionized calcium-binding adapter molecule, MBP=myelin basic protein.

FIG. 17 is a diagram of coronal mouse brain section indicating cell injection site within the corpus callosum adjacent to the stroke lesion. Blue arrows indicate the approximate injection sites for L-NIO to induce the stroke lesion. Black arrow indicates the approximate location of the cell injection site adjacent to the stroke lesion core (red area). The approximate lesion core is shown in blue. Abbreviations: Cx=cortex; Str=striatum; WM=white matter.

FIGS. 18A-18E show representative fluorescent photomicrographs of astrocyte and microglia/immune cell activation in the mouse brain following stroke injury and AST-OPC1 transplantation adjacent to the lesion core. Image panels show fluorescent immunostaining of astrocytes (GFAP) and activated microglia/immune cells (IBA-1) in the uninjured contralateral hemisphere (FIGS. 18A-18B) and stroke-injured/AST-OPC1 transplanted hemisphere (FIGS. 18C-18E) three weeks post-stroke and two weeks post-AST-OPC1 transplantation. In FIG. 18A and FIG. 18B, the left panel shows the merged image for GFAP (green) and IBA-1 (orange), the middle panel shows GFAP alone, and the right panel shows IBA-1 alone. The white box in FIG. 18A indicates the magnified region shown in FIG. 18B. In FIGS. 18C-18D, the left panel shows IBA-1 alone, the middle panel shows GFAP alone, and the right panel shows the merged image. In FIG. 18E, the left panel shows the merged image for GFAP (green) and IBA-1 (orange), the middle panel shows GFAP alone, and the right panel shows IBA-1 alone. The white boxes in FIG. 18C and FIG. 18D indicate the magnified regions shown in FIG. 18D and FIG. 18E, respectively. The asterisks in FIG. 18D and FIG. 18E indicate a common landmark between the image panels. Magnifications: FIG. 18A and FIG. 18C=40×, FIG. 18B=600×, FIG. 18D=100×, and FIG. 18E=1000×. Abbreviations: Cx=cortex; GFAP=glial fibrillary acid protein; IBA-1=ionized calcium-binding adapter molecule; Str=striatum.

FIGS. 19A-19B show representative fluorescent photomicrographs of AST-OPC1 location, reactive astrocytes, and activated microglia/immune cells in the mouse brain following stroke injury and AST-OPC1 transplantation adjacent to the lesion core. Image panels show fluorescent immunostaining of AST-OPC1 (Hnuc) and reactive astrocytes (GFAP) and activated microglia/immune cells (IBA-1) in the uninjured contralateral hemisphere (FIG. 19A) and the stroke-injured/AST-OPC1 transplanted hemisphere (FIG. 19B) three weeks post-stroke and two weeks post-AST-OPC1 transplantation. In FIG. 19A and FIG. 19B panel 1 (left-most) shows the merged image for Hnuc (blue), GFAP (green), and IBA-1(red) within the corpus callosum (as indicated by the diagram between FIG. 19A and FIG. 19B). Magnification=600×. Abbreviations: GFAP=glial fibrillary acid protein; Hnuc=anti-human nuclei; IBA-1=ionized calcium-binding adapter molecule.

FIGS. 20A-20B show representative fluorescent photomicrographs of AST-OPC1 location, reduced myelin loss, and endogenous neurogenesis in the mouse brain following stroke injury and AST-OPC1 transplantation adjacent to the lesion core. FIG. 20A shows fluorescent immunostaining of AST-OPC1 (Hnuc) and myelin (MBP) and immature neurons (DCX) in the stroke-injured/AST-OPC1 transplanted hemisphere three weeks post-stroke and two weeks post-AST-OPC1 transplantation. Left-most panels show the merged image for MBP (green) and DCX (red) (Top and middle panels) or Hnuc (blue) and MBP (green) bottom panel). The center column of panels shows single staining for MBP or Hnuc, and the right column panels show single staining for DCX or MBP. FIG. 20B is a diagram indicating the region shown in FIG. 20A. Magnifications: (Top row)=200×, (Middle row)=600×, (Bottom row)=1000×. Abbreviations: DCX=doublecortin; Hnuc=anti-human nuclei; MBP=myelin basic protein.

FIGS. 21A-21D show representative fluorescent photomicrographs of microglia/immune cell activation, myelin loss, and oligodendrocyte response in the mouse brain following stroke injury and AST-OPC1 transplantation adjacent to the lesion core. Image panels show fluorescent immunostaining of activated microglia/immune cells (IBA-1), myelin (MBP), and oligodendrocytes (OLIG2) in the uninjured contralateral hemisphere (FIG. 21A, FIG. 21C) and the stroke-injured/AST-OPC1 transplanted hemisphere (FIG. 21B, FIG. 21D) three weeks post-stroke and two weeks post-AST-OPC1 transplantation. In FIG. 21A, panel 1 (left-most) shows the merged image for IBA-1 (blue), MBP (green), and OLIG2 (red) within the contralateral corpus callosum. Panel 2 shows IBA-1 alone, panel 3 shows MBP alone, and panel 4 shows OLIG2 alone. In FIG. 21B, panel 1 (left-most) shows OLIG2 alone, panel 2 shows MBP alone, panel 3 shows IBA-1 alone, and panel 4 shows the merged image of all three stains. FIG. 21C and FIG. 21D show the merged images for IBA-1 (blue) and OLIG2 (red) only. The diagram between panels FIG. 21A and FIG. 21B indicates the regions shown in the fluorescent micrographs. Magnification=200×. Abbreviations: IBA-1=ionized calcium-binding adapter molecule, MBP=myelin basic protein.

FIGS. 22A-22D show representative fluorescent photomicrographs of AST-OPC1 location in the mouse brain following stroke injury and AST-OPC1 transplantation adjacent to the lesion core. Image panels show fluorescent immunostaining of AST-OPC1 (Hnuc, blue) in the uninjured contralateral hemisphere (FIG. 22A, FIG. 22C) and the stroke-injured/AST-OPC1 transplanted hemisphere (FIG. 22B, FIG. 22D) three weeks post-stroke and two weeks post-AST-OPC1 transplantation. The diagram in the center of the figure indicates the regions shown in FIGS. 22A-22D. Magnification=200×. Abbreviations: Hnuc=anti-human nuclei.

FIGS. 23A-23B show quantification of infarct volume and glial scar volume in the mouse brain following stroke injury and AST-OPC1 transplantation. FIG. 23A shows mean infarct volume for each treatment group at 3 weeks post-stroke (or sham surgery) and 2 weeks post-AST-OPC1 transplantation. FIG. 23B shows mean glial scar volume for each treatment group at 3 weeks post-stroke (or sham surgery) and 2 weeks post-AST-OPC1 transplantation. Treatment group labels: WMS=white matter stroke alone; OPC=sham surgery plus AST-OPC1 transplant; WMS+OPC ins=stroke plus AST-OPC1 transplantation inside the stroke lesion; WMS+OPC out=stroke plus AST-OPC1 transplantation adjacent to the stroke lesion. Error bars denote standard error of the mean.

FIGS. 24A-24B show quantification of myelin basic protein immunoreactivity and oligodendrocyte response within the corpus callosum lesion following stroke injury and AST-OPC1 transplantation. FIG. 24A shows mean myelin basic protein (MBP) immunoreactivity for each treatment group at 3 weeks post-stroke (or sham surgery) and 2 weeks post-AST-OPC1 transplantation. FIG. 24B shows the average number of OLIG2 positive cells for each treatment group at 3 weeks post-stroke (or sham surgery) and 2 weeks post-AST-OPC1 transplantation. Treatment group labels: Naïve=uninjured, non-transplanted; WMS=white matter stroke alone; OPC=sham surgery plus AST-OPC1 transplant; WMS+OPC ins=stroke plus AST-OPC1 transplantation inside the stroke lesion; WMS+OPC out=stroke plus AST-OPC1 transplantation adjacent to the stroke lesion. Error bars denote standard error of the mean.

FIG. 25 shows the experimental timeline of Study 2 testing the effects of AST-OPC1 on functional recovery and white matter sparing in a mouse model of white matter stroke. Key time points in the experimental design are shown, including AST-OPC1 transplantation 7 days post-stroke and monthly behavior testing. AST-OPC1 dose=100,000 cells/mouse as a single 1 μL injection.

FIG. 26 shows AST-OPC1 transplantation adjacent to the lesion site improves performance of stroke-injured mice in the gridwalking test. Mean performance in the gridwalking test (shown as % foot faults) is shown for each treatment group as a function of time (post-stroke). One week post-stroke corresponds to the day prior to AST-OPC1 transplantation. Asterisk and hashtag indicate significance relative to the uninjured control group using two-way ANOVA with Tukey's HSD post-hoc analysis (p<0.05). At 4 months post-stroke, all stroke-injured groups (+/−AST-OPC1) were significantly different from control, uninjured animals, indicating sustained deficit. At 6 months post-stroke, only the stroke alone group and the stroke group administered high dose AST-OPC1 inside the infarct (stroke+cells High dose Inside) were significantly different from control, uninjured animals, indicating positive recovery in the other stroke-injured AST-OPC1 treatment groups. Treatment group labels: Control=uninjured, non-transplanted; stroke=stroke alone; stroke+cells high dose inside=stroke injury+100,000 AST-OPC1 cells transplanted inside the stroke lesion; stroke+cells high dose outside=stroke injury+100,000 AST-OPC1 cells transplanted adjacent to the stroke lesion; stroke+cells low dose outside=stroke injury+10,000 AST-OPC1 cells transplanted adjacent to the stroke lesion; cells high dose=100,000 AST-OPC1 cells transplanted into the uninjured corpus callosum; cells low dose=10,000 AST-OPC1 cells transplanted into the uninjured corpus callosum.

FIG. 27 shows AST-OPC1 transplantation adjacent to the lesion site improves performance of stroke-injured mice in the cylinder test. Mean performance in the cylinder test (shown as motor deficit relative to pre-injury baseline) is shown for each treatment group as a function of time (post-stroke). One week post-stroke corresponds to the day prior to AST-OPC1 transplantation. Asterisk indicates significance relative to the stroke alone group using two-way ANOVA with Tukey's HSD post-hoc analysis (p<0.05). At 4 months post-stroke, all stroke-injured groups that received AST-OPC1 were significantly different from the stroke only animals, indicating improved performance following AST-OPC1 treatment. Treatment group labels: Control=uninjured, non-transplanted; stroke=stroke alone; stroke+cells high dose inside=stroke injury+100,000 AST-OPC1 cells transplanted inside the stroke lesion; stroke+cells high dose outside=stroke injury+100,000 AST-OPC1 cells transplanted adjacent to the stroke lesion; stroke+cells low dose outside=stroke injury+10,000 AST-OPC1 cells transplanted adjacent to the stroke lesion; cells high dose=100,000 AST-OPC1 cells transplanted into the uninjured corpus callosum; cells low dose=10,000 AST-OPC1 cells transplanted into the uninjured corpus callosum.

FIG. 28 shows representative photomicrographs of Nissl-stained mouse brain following white matter stroke injury and AST-OPC1 transplantation. Image panels show representative Nissl staining of the corpus callosum and lateral ventricle in the lesioned (left) and uninjured, contralateral (right) hemispheres for each treatment group. Increased tissue sparing in the corpus callosum and reduced enlargement of the ipsilateral lateral ventricle is evident in the AST-OPC1 treatment groups, most notably in the stroke-injured group administered high dose AST-OPC1 adjacent to the lesion site. Treatment group labels: Control=uninjured, non-transplanted; white matter stroke=stroke alone; stroke+cells high dose inside=stroke injury+100,000 AST-OPC1 cells transplanted inside the stroke lesion; stroke+cells high dose outside=stroke injury+100,000 AST-OPC1 cells transplanted adjacent to the stroke lesion; stroke+cells low dose outside=stroke injury+10,000 AST-OPC1 cells transplanted adjacent to the stroke lesion.

### DETAILED DESCRIPTION

Before the present compositions and methods are described, it is to be understood that the inventions disclosed herein are not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Any methods and materials similar or equivalent to those described herein can be used in the practice or testing of embodiments of the present disclosure.

In various embodiments, the methods and compositions described herein pertain to the discovery that AST-OPC1 transplantation after cerebral ischemic injury enhances recovery in a murine model of WMS. Transplantation of AST-OPC1 at subacute time points (7 days after stroke) directly adjacent or inside the lesion core produced widespread migration of AST-OPC1 throughout subcortical white matter and resulted in increased myelination within the damaged white matter and reduced measures of reactive astrocytosis and inflammation. MRI imaging of white matter after transplantation of AST-OPC1 directly adjacent to the lesion core showed reduction in the hyperintensities that are characteristic of white matter damage in both the mouse model and human WMS. Behavioral evaluation demonstrated improvements in two tests of motor function. These results indicate that AST-OPC1 transplantation promotes white matter repair and recovery in WMS.

In various embodiments methods for the use of AST-OPC1 in the treatment of cerebral ischemic injury, such as white matter stroke are provided. Also provided herein are pharmaceutical compositions and formulations suitable for use in cell-based clinical therapy of white matter stroke.

### Uses of the Cells of the Invention

Methods to produce large numbers of highly pure, characterized oligodendrocyte progenitor cells from human embryonic stem cells have been described previously (e.g., U.S. provisional patent applications 62/162,739 and 62/144,921). Derivation of oligodendrocyte progenitor cells (OPCs) from human embryonic stem cells a provides a renewable and scalable source of OPCs for a number of important therapeutic, research, development, and commercial purposes, including treatment of cerebral ischemic injuries. It is also recognized that oligodendrocyte progenitor cells can be derived from sources other than human embryonic stem cells. Such sources include, but are not limited to adult stem cells, induced pluripotent stem cells IPSCs, cultured stem cell lines, and the like.

The term “AST-OPC1” refers to a specific, characterized, in vitro differentiated cell population containing a mixture of oligodendrocyte progenitor cells (OPCs) and other characterized cell types obtained from undifferentiated stem cells according to the specific differentiation protocols disclosed herein.

Compositional analysis of AST-OPC1 by immunocytochemistry (ICC), flow cytometry, and quantitative polymerase chain reaction (qPCR) demonstrates that the cell population is comprised primarily of neural lineage cells of the oligodendrocyte phenotype. Other neural lineage cells, namely astrocytes and neurons, are present at low frequencies. The only non-neural cells detected in the population are epithelial cells. Mesodermal, endodermal lineage cells and uhESCs are routinely below quantitation or detection of the assays.

The term “oligodendrocyte progenitor cells” (OPCs) as used herein, refers to cells of neuroectoderm/glial lineage having the characteristics of a cell type found in the central nervous system, capable of differentiating into oligodendrocytes. These cells typically express the characteristics markers Nestin, NG2, and PDGR-Ra.

The terms “treatment,” “treat” “treated,” or “treating,” as used herein, can refer to both therapeutic treatment or prophylactic or preventative measures, wherein the object is to prevent or slow down (lessen) an undesired physiological condition, symptom, disorder or disease, or to obtain beneficial or desired clinical results. In some embodiments, the term may refer to both treating and preventing. For the purposes of this disclosure, beneficial or desired clinical results may include, but are not limited to one or more of the following: alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response. Treatment also includes prolonging survival as compared to expected survival if not receiving treatment. In certain embodiments, particularly in the case of cerbreal ischemia, treatment may include improving or restoring motor control, improving or restoring speech, improving or restoring balance, improving cognition (e.g., as measured by any of a vaiety of cognitive function assays), and the like.

The term “subject,” as used herein includes, but is not limited to, humans, non-human primates and non-human vertebrates such as wild, domestic and farm animals including any mammal, such as cats, dogs, cows, sheep, pigs, horses, rabbits, rodents such as mice and rats. In some embodiments, the term “subject,” refers to a male. In some embodiments, the term “subject,” refers to a female.

AST-OPC1 promotes myelin repair or remyelination in human patients or other subjects in need of cell therapy. The following are non-limiting examples of conditions, diseases and pathologies requiring myelin repair or remyelination: brain ischemic injuries including white matter stroke, multiple sclerosis, the leukodystrophies, the Guillain-Barre Syndrome, the Charcot-Marie-Tooth neuropathy, Tay-Sachs disease, Niemann-Pick disease, Gaucher disease, Hurler syndrome and traumatic injuries resulting in loss of myelination, such as acute spinal cord injury.

In addition to myelin repair or remyelination, AST-OPC1 secretes neurotrophic factors, e.g. BDNF, that may directly provide reparative action on the ischemic tissue.

The OPCs are administered in a manner that permits them to reside at, graft to, and/or migrate to the intended tissue site. Administration of the cells to a subject may be achieved by any method known in the art. For example the cells may be administered surgically directly to the organ or tissue in need of a cellular transplant. Alternatively non-invasive procedures may be used to administer the cells to the subject. Examples of non-invasive delivery methods include the use of syringes and/or catheters and/or cannula to deliver the cells into the organ or tissue in need of cellular therapy.

In certain embodiments, the OPCs are administered into the infarct core. In other embodiments, the OPCs are administered directly adjacent to the infarct core. “Directly adjacent”, as used herein, refers to the area outside the infarct core that in some instances represents an area of partial stroke damage; in other instances, “directly adjacent” refers to healthy tissue outside the infarct region. In some embodiments, the OPCs are administered 0.01 mm to 10 mm from the infarct core. In some embodiments, the OPCs are administered 0.05 mm to 3 mm from the infarct core. In some embodiments, the OPCs are administered 0.1 mm to 2 mm from the infarct core. In some embodiments, the OPCs are administered 0.5 mm to 1 mm from the infarct core. In some embodiments, the OPCs are administered 0.3 mm to 0.6 mm from the infarct core.

In certain embodiments, the OPCs are administered to the subject during the subacute time period. “Subacute” as used herein refers to the time period between acute and chronic phases during which the initial damage and cell death from the stroke injury has ended. As used herein, “early subacute” in a human subject refers to up to one month after the stroke and “late subacute” refers to the time period 1-3 months after the stroke.

In certain embodiments, the subject receiving OPCs of the invention can be treated to reduce immune rejection of the transplanted cells. Methods contemplated include, but are not limited to the administration of traditional immunosuppressive drugs like tacrolimus, cyclosporin A (Dunn et al., Drugs 61:1957, 2001), or inducing immunotolerance using a matched population of pluripotent stem derived cells (WO 02/44343; U.S. Pat. No. 6,280,718; WO 03/050251). In certain embodiments, a combination of anti-inflammatory (such as prednisone) and immunosuppressive drugs may be used. In certain embodiments, the OPCs can be supplied in the form of a pharmaceutical composition, comprising an isotonic excipient prepared under sufficiently sterile conditions for human administration.

For general principles in medicinal formulation, the reader is referred to Allogeneic Stem Cell Transplantation, Lazarus and Laughlin Eds. Springer Science+Business Media LLC 2010; and Hematopoietic Stem Cell Therapy, E. D. Ball, J. Lister & P. Law, Churchill Livingstone, 2000. Choice of the cellular excipient and any accompanying elements of the composition will be adapted in accordance with the route and device used for administration. The composition may also comprise or be accompanied with one or more other ingredients that facilitate the engraftment or functional mobilization of the enriched target cells. Suitable ingredients may include matrix proteins that support or promote adhesion of the target cell type or that promote vascularization of the implanted tissue.

### Pharmaceutical Compositions of the Invention

The oligodendrocyte progenitor cells may be administered to a subject in need of therapy per se. Alternatively, the cells may be administered to the subject in need of therapy in a pharmaceutical composition mixed with a suitable carrier and/or using a depot delivery system.

As used herein, the term “pharmaceutical composition” refers to a preparation comprising a therapeutic agent or therapeutic agents in combination with other components, such as physiologically suitable carriers and excipients.

As used herein, the term “therapeutic agent” refers to the cells of the invention accountable for a biological effect in the subject. Depending on the embodiment of the invention, “therapeutic agent” may refer to the oligodendrocyte progenitor cells of the invention. Additionally or alternatively, “therapeutic agent” may refer to one or more factors secreted by the oligodendrocyte progenitor cells of the invention with a role in aiding neural repair.

As used herein, the term “therapeutically effective amount” means a dosage, dosage regimen, or amount sufficient to produce a desired result.

As used herein, the terms “carrier” “physiologically acceptable carrier” and “biologically acceptable carrier” may be used interchangeably and refer to a diluent or a carrier substance that does not cause significant adverse effects or irritation in the subject and does not substantially abrogate the biological activity or effect of the therapeutic agent. The term “excipient” refers to a substance added to a pharmaceutical composition to further facilitate administration of the therapeutic agent.

The compositions of the present invention can be formulated for parenteral administration by injection, e.g., by bolus injection or continuous infusion. The compositions can be administered by continuous infusion subcutaneously over a period of about 15 minutes to about 24 hours. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

The therapeutic agents described herein may be administered as a component of a hydrogel, such as those described in U.S. patent application Ser. No. 14/275,795, filed May 12, 2014, and U.S. Pat. Nos. 8,324,184 and 7,928,069. Hydrogels comprising synthetic polymers such as poly (hydroxyethyl methacrylate) (PHEMA), poly-(ethylene glycol) (PEG) and poly (vinyl alcohol) (PVA) and/or comprising naturally sourced material such as collagen, hyaluronic acid (HA), fibrin, alginate, agarose and chitosan are known in the art (Peppas et al. (2006) Advanced Materials 18:1345; Lee et al. (2001) Chem. Rev. 101:1869). Covalently cross-linked hydrogels formed by various chemical modifications have also been previously described (Vercruysse et al. (1997) Bioconjugate Chem. 8:686; Prestwich et al. (1998) J. Controlled Release 53:93; Burdick et al. (2005) Biomacromolecules 6:386; Gamini et al. (2002) Biomaterials 23:1161; U.S. Pat. No. 7,928,069; U.S. Pat. No. 7,981,871).

Hydrogels based on thiol-modified derivatives of hyaluronic acid (HA) and porcine gelatin cross-linked with polyethylene glycol diacrylate (PEGDA) (trade name HyStem®) have unique chemical, biological and physical attributes making them suitable for many applications including cell culture, drug delivery and the like (Shu et al. (2004) J of Biomed Mat Res Part A 68:365; Shu et al. (2002) Biomacromolecules 3:1304; Vanderhooft et al. (2009) Macromolecular Biosci 9:20. Cross-linked HA hydrogels, including HyStem®, have been successfully used in animal models of corneal epithelial wound healing Yang et al. (2010) Veterinary Opthal 13:144, corneal tissue engineering (Espandar et al. (2012) Archives of Opthamol 130:202, and retinal repair Liu et al. (2013) Tissue Engineering Part A 19:135.

The preclinical use of hydrogels to maintain bioactivity and slow release of biologics has been described (Cai et al. (2005) Biomaterials 26:6054; Zhang (2011) Biomaterials 32:9415; Overman et al. (2012) Proceedings of the National Academy of Sciences of the United States of America 109:E2230; Garbern et al. (2011) Biomaterials 32:2407; Koutsopoulos et al. (2009) Proceedings of the National Academy of Sciences of the United States of America 106:4623. Furthermore, their use in cell delivery has been shown to improve cell viability and localization post-implantation (Laflamme et al. (2007) Nature Biotechnology 25:1015; Zhong et al. (2010) Neurorehabilitation and Neural Repair 24:636; Compte et al. (2009) Stem Cells 27:753. Several different hydrogels have been used as excipients in FDA-approved ocular small molecule therapeutics to increase their residence time on the eye surface (Kompella et al. (2010) Therapeutic Delivery 1:435.

In addition, two new hydrogel formulations have been reported that show promise in delivering therapeutic cells (Ballios et al. (2010) Biomaterials 31:2555; Caicco et al. (2012) Journal of Biomedical Materials Research Part A 101:1472; Yang et al. (2010) Veterinary ophthalmology 13:144; Mazumder et al. (2012) Journal of Biomedical Materials Research Part A 100:1877.

### Materials and Methods

Animal Subjects.

All procedures used were approved by a board-certified veterinarian and were conducted in accordance with the National Institute of Health Guide for the Care and Use of Laboratory Animals. NSG mice (Shultz et al., (2007) Nat Rev Immunol. 7(20:118; jaxmice.jax.org/nod-scid-gamma) were obtained from Jackson Laboratories (Bar Harbor, Me.). All animal subjects were housed in standard conditions with a 12 hr light/dark cycle and were provided food and water ad libitum.

Induction of Focal Ischemic Lesions Using L-Nio.

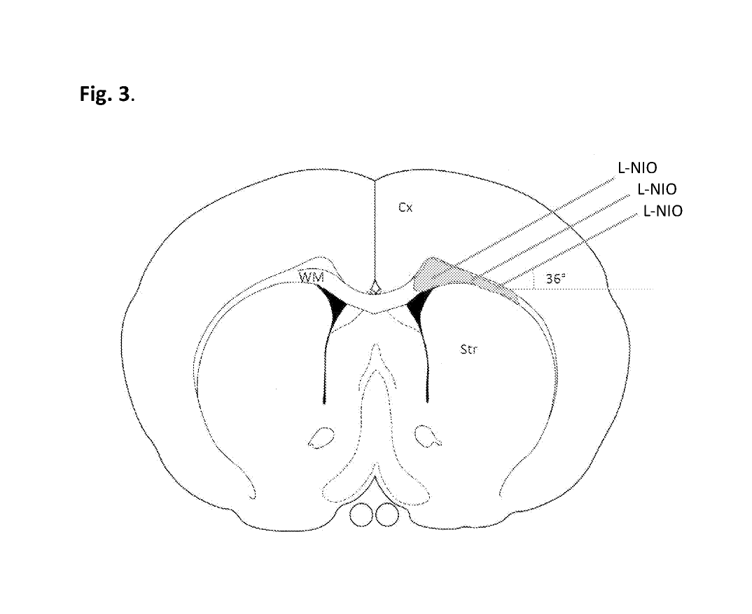

A previously established mouse model of subcortical white matter stroke (Sozmen et al., (2009) J. Neurosci Methods 180(2):261; Hinman et al., (2013) Stroke 44(1):182) that mimics the large white matter lesions seen in moderate to advanced human white matter ischemia or vascular dementia was used. Briefly, to induce focal ischemic lesions, N5-(1-iminoethyl)-L-ornithine, dihydrochloride (L-Nio, Calbiochem), was injected at three stereotactic coordinates directly into the corpus callosum of each mouse brain, as illustrated in FIG. 3.

Differentiation of AST-OPC1 from hESCs.

The WA01 (H1) hESC line was expanded in feeder-free conditions. hESC colonies were lifted with collagenase and manual scraping and then seeded into ultra-low attachment flasks (Day 0) in 50% hESC growth media and 50% glial progenitor medium (GPM) containing 4 ng/mL of basic fibroblast growth factor (FGF) and 20 ng/mL epidermal growth factor (EGF) to stimulate embryoid body formation. On Day 1, media was replaced with 50% hESC growth media/50% GPM containing 20 ng/mL EGF and 10 μM all-trans-retinoic acid (RA). On Days 2-8, media was replaced daily with 100% GPM containing 20 ng/mL EGF and 10 μM RA. On Days 9-26, embryoid bodies were maintained in GPM/EGF media without RA, and media was replaced every 2 days. On Day 28, embryoid bodies were plated in Matrigel-coated flasks and cultured in GPM/EGF media for 7 days with media exchange every 2 days. On Day 34, cells were harvested with trypsin, replated in Matrigel-coated flasks, and cultured for an additional 7 days in GPM/EGF media, with media exchange every 2 days. On Day 41, cells were harvested with trypsin, filtered to remove residual cell aggregates, and cryopreserved in liquid nitrogen.

Analysis of Differentiated AST-OPC1 by Flow Cytometry.

Differentiated AST-OPC1 samples were assayed for the presence of surface and intracellular markers using standard flow cytometry. For surface marker staining, Day 41 AST-OPC1 samples were blocked with 10% heat-inactivated goat serum (HI FBS) and then incubated with primary antibody and/or isotype control (0.5 μg/5×10⁵ cells, NG2, Invitrogen #37-2300; Mouse IgG1 BD Biosciences #55412) for 30 minutes at 2-8° C., washed, then and incubated with secondary antibody (goat-anti-mouse-IgGl-A488 Invitrogen A21121 at 0.25 μg/5×10⁵ cells) for 30 minutes at 2-8° C. To exclude nonviable cells, propidium iodide (Sigma P4864 at 1 μg/mL) was added to the stained samples just prior to acquisition. All samples were then acquired and the data analyzed on the BD Biosciences FACSCalibur™ cytometer system using Cellquest Pro software.

For intracellular marker staining, Day 41 AST-OPC1 samples were tagged with ethidium monoazide (Sigma E2028 at 5 μg/mL) for dead cell discrimination followed by fixation using 2% Paraformaldehyde (PFA) and then permeabilization with cold 90% methanol. The cells were blocked with either 5% HI FBS (for Oct4) or 10% heat-inactivated goat serum (for Nestin) and then incubated with primary antibody and/or isotype control (goat anti-Oct4 Santa Cruz SC8629, normal goat IgG SC2028 at 0.15-0.5 μg/5×10⁵ cells; Nestin Millipore MAB5326; MoIgG1 BD Biosciences 554121 at 0.5 μg/5×10⁵ cells) for 30 minutes at 2-8° C., washed with stain buffer and incubated with secondary antibody (donkey-anti-goat-IgG-A488 Invitrogen A11055 or goat-anti-mouse-IgG1-A488 Invitrogen A21121 at 0.25 μg/5×10⁵ cells) for 30 minutes at 2-8° C. All samples were then acquired and the data analyzed on the BD Biosciences FACSCalibur™ cytometer system using Cellquest Pro software.

AST-OPC1 Transplantation in NSG Mice.

AST-OPC-1 cells were received in dry ice from Asterias Biotheraputics, Inc and the “AST-OPC1 Dose Preparation Procedure” (AST-GEN-0021 version 0.2) protocol was followed. Five different groups of animals received AST-OPC1. Cells were stereotaxically transplanted seven days after stroke. The temperature of the mice were monitored and maintained at 36.5-37.5° C. using a rectal probe and heating pad. A Hamilton syringe was filled with AST-OPC1 secured onto the stereotaxic arm and connected to a pressure pump. An incision was made at the coordinates shown on Table 1, at an angle of 36°, and two injections of cells were given. The high dose group received a total of approximately 100,000 cells in one μL, and low dose group received a total of approximately 10,000 cells in one μL. The needle was left in situ for 2 minutes after the first injection, and for 4 minutes after the second injection.

TABLE 1
AST-OPC1 transplantation coordinates.

| Treatment groups | AP | ML | DV |
|---|---|---|---|
| Stroke + High dose of cells inside infarct | +0.14 | +3 | −1.32 |
| Stroke + Low dose of cells inside infarct | +0.14 | +3 | −1.32 |
| Stroke + Low dose of cell outside infarct | +0.14 | +2 | −1.4 |
| Cells only high dose | +0.14 | +3 | −1.32 |
| Cells only low dose | +0.14 | +3 | −1.32 |

AP = Anterior-Posterior;
ML = Medial-Lateral;
DV = Dorsal-Ventral.

Brain Tissue Processing for Immunofluorescence.

After the post-surgery survival period (15 days), each mouse was given an overdose of isoflurane and perfused transcardially with 0.1M phosphate buffered saline followed by 4% paraformaldehyde. The brains were removed, postfixed overnight in 4% paraformaldehyde and cryoprotected for 2 days in 30% sucrose. Subsequently brains were removed and frozen. Brain tissue was sectioned into 40 μm sections 200 μm apart using a cryostat (Leica CM 0530).

Immunostaining for human nuclear antigen (HuNu), the microglial/macrophage marker IBA-1, the neuronal marker NF200, the astrocyte marker GFAP, the pan-oligodendrocyte marker Olig2, the mature oligodendrocyte marker MBP and the immature neuronal marker DCx was done by blocking in 5% normal donkey serum for 1 hour at room temperature, incubation in primary antibody overnight at 4 degrees Celsius, incubation in secondary antibody for 1 hour at room temperature, mounting sections onto subbed slides and air drying. Mounted sections were then dehydrated, in ascending concentrations of alcohol and xylene, and cover slipped with DPX. Primary antibodies were: mouse anti-HuNu (1:150, Millipore), rabbit anti-Iba-1 (1:500, Wako Chemicals), rabbit anti-NF200 (1:500, Sigma), rat anti-myelin basic protein (MBP, 1:500, Millipore), rabbit anti-Olig2 (1:500, Millipore), rat anti-GFAP (1:500, Millipore), goat anti-doublecortin (1:500, Santa Cruz Biotechnologies) and mouse monoclonal-hNuc (1:100, Millipore). All secondary antibodies were donkey F(ab′)2 fragments conjugated to Cy2(cyan) or Cy3(yellow) (Jackson Immunoresearch) dyes and were used at a dilution of 1:1000.

High-resolution confocal images in Z-stacks were acquired (Nikon C2 confocal system). Area measurements of the infarct core, IBA-1, GFAP, HuNu, DCX and Olig2 positive cells were stereologically quantified using the optical fractionator probe and neuroanatomical quantification software (Stereoinvestigator, MBF Bioscience). White matter axonal projections stained with NF200 and MBP were quantified with intensity profiles (ImageJ, NIH).

Nissl Staining.

Nissl staining was used for the histological examination and measurement of neuronal loss. Slides with 40 μm brain sections were dehydrated in graded ethanol (50%, 70%, 95% and 100%), washed on 50% ethanol-50% chloroform for 1 hour and rehydrated in graded ethanol (100%, 95%, 70%, 50%). The sections were stained with a cresyl violet solution for 45 seconds, rinsed with distilled water, washed in 70% ethanol containing a few drops of glacial acetic acid and dehydrated in graded ethanol (70%, 95% and 100%), placed in xylene and cover slipped using mounting medium. Stained sections were visualized on a Leica DM LB florescent microscope.

Magnetic Resonance Imaging (MRI).

Mice were anesthetized and placed in a Bruker 7T small animal MRI (Bruker Biospin, Switzerland). MRI imaging was performed on days 0, 7 and 6 months after stroke. Respiratory rate was monitored throughout the procedure and body temperature was maintained at 37±0.5° C. A T2-weighted image set was acquired: rapid acquisition relaxation enhancement factor 8, repetition time 5300 ms, echo time 15.00 ms with an in-plane resolution of 0.01.56_0.0156_0.50 mm with 13 contiguous slices.

Tractography.

Tractography, diffusion tensor data (DTI) were acquired at 0, 7 days and 6 months after treatment with a spin echo single shot echo planar imaging (EPI) pulse sequence using the following parameters: TR/TE: 5000/35 ms; a signal average of 10, a 30 noncolinear diffusion gradient scheme with diffusion weighting of b=1000 s/mm2 and b=0 s/mm2, and field of view 3.5×3.5 cm. The data was acquired using 30 directions with a single shot EPI sequence on a 96×96 matrix, and zero-filled k-space to construct a 128×128 image matrix. The images were obtained with medInria, a multi-platform medical image processing and visualization software. DTI tractography data was performed in the lesion zone using n=6 animals per group. Zoomed lesion site 3D views of DTI tractography images are represented using ParaView 4.1.0 software

### EXAMPLES

The following examples are not intended to limit the scope of what the inventors regard as their invention nor are they intended to represent that the experiments below are all or the only experiments performed.

### Example 1: Derivation and Characterization of AST-OPC1

AST-OPC1 (formerly known as GRNOPC1) was generated by the differentiation of WA01 (H1) hESCs from a master cell bank (MCB) as described in the Materials and Methods. The differentiation process to produce AST-OPC1 requires 41 days and transitions the hESCs from undifferentiated cell colonies through embryoid bodies to become an adherent, dispersed cell population which is harvested and cryopreserved.

Analysis of 41 day differentiated AST-OPC1 by flow cytometry and immunocytochemistry (ICC) has been previously described (U.S. provisional patent applications 62/162,739 and 62/144,921). The ICC and flow cytometry results indicated that the cell population was comprised mostly of neural lineage cells of the early oligodendrocyte progenitor phenotype. By flow cytometry, over 90% of the cells were positive for Nestin and >50% were positive for NG2, a neural/glial proteoglycan expressed by oligodendrocyte progenitor cells. In addition, levels of the pluripotent stem cell marker, Oct4, were below the level of quantitation (<0.2%), indicating a lack of residual hESCs. Using an alternative high content image analysis assay, we further determined that the frequency of Oct4+ cells in AST-OPC1 was less than 0.05%. Using the defined AST-OPC1 differentiation process, we produced over 75 lots of AST-OPC1, which we further characterized by ICC on day 41 for the presence of multiple markers of ectodermal, mesodermal, endodermal, and pluripotent cell types to assess the composition of the population and detect potential unwanted cells types. In agreement with the flow cytometry results, ICC profiling indicated a cell population predominantly composed of early oligodendrocyte progenitor cells with few mature neuronal or astrocytic cells. The presence of endodermal, mesodermal or pluripotent cell types was undetectable to <1% of the differentiated AST-OPC1 cell population.

### Example 2: Study 1—AST-OPC1 Transplantation in a NSG Murine Model of White Matter Stroke

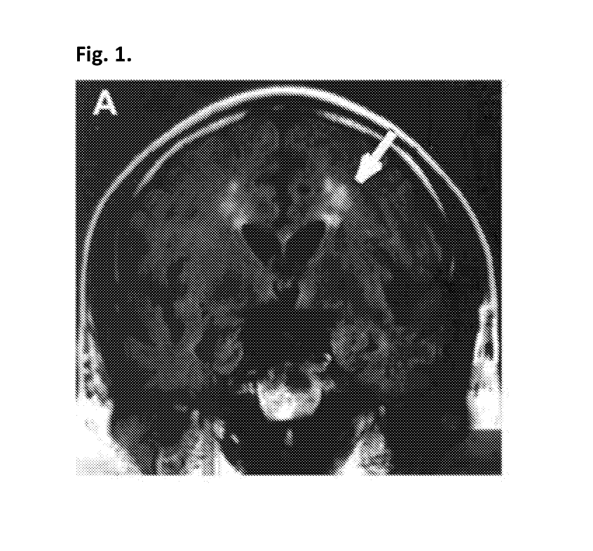

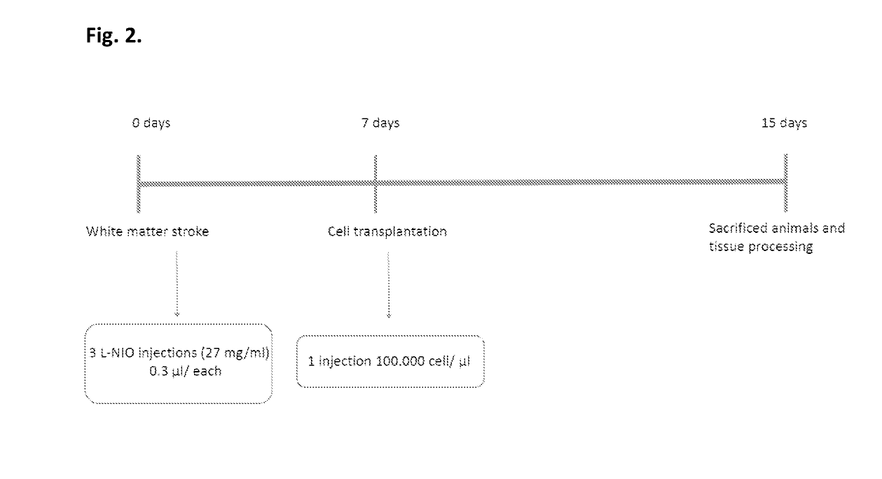

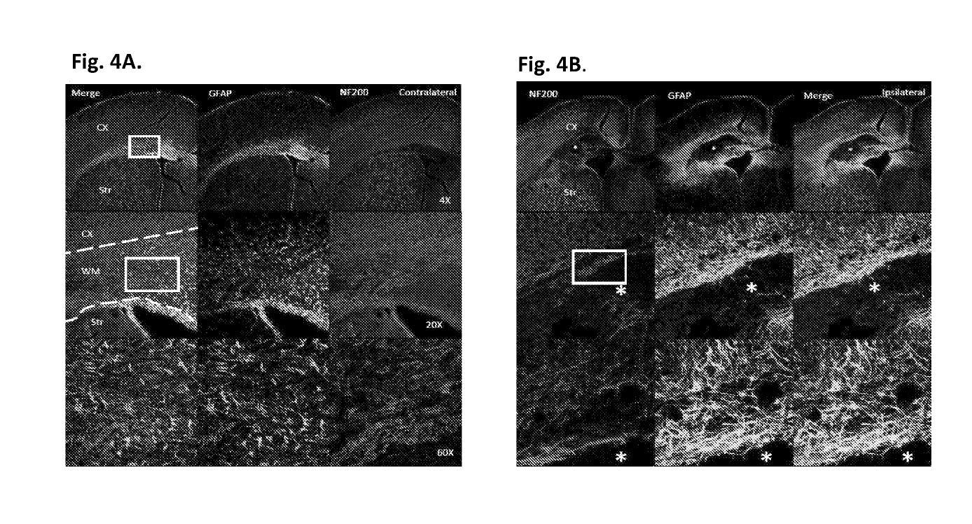

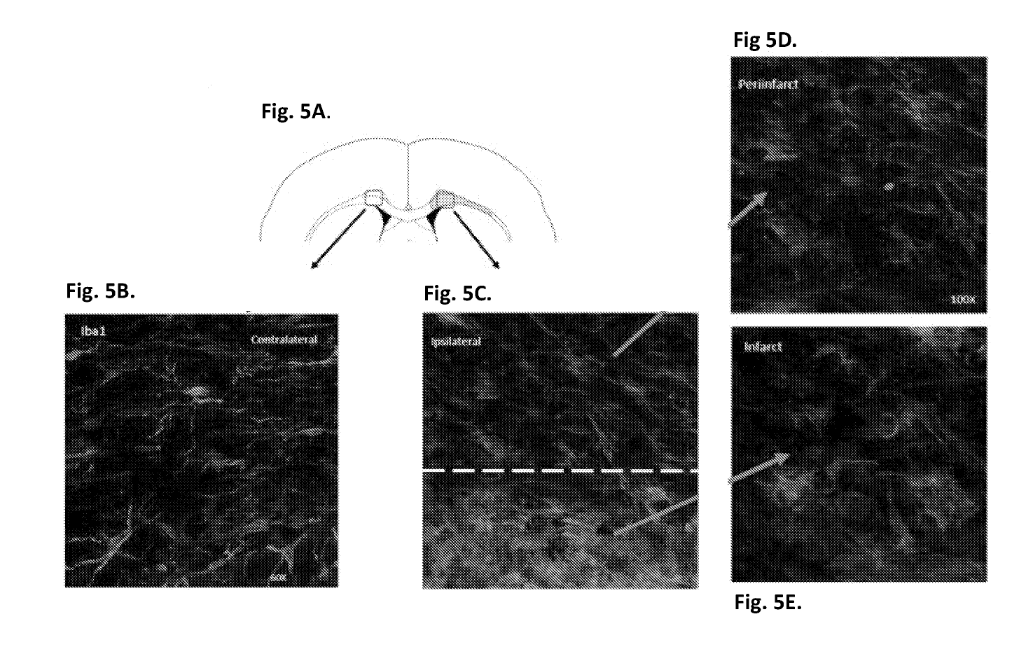

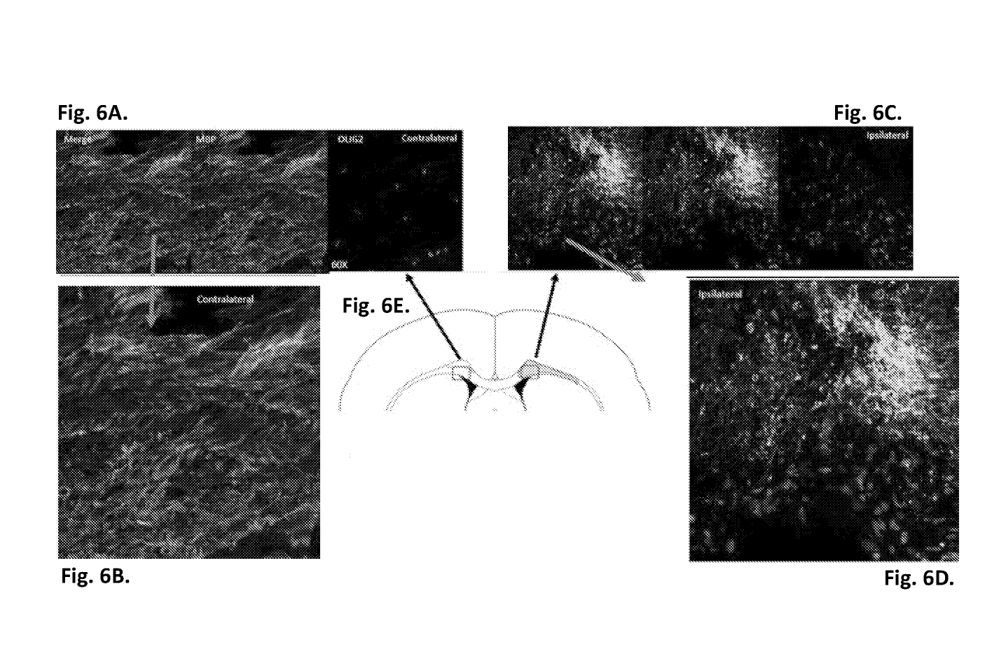

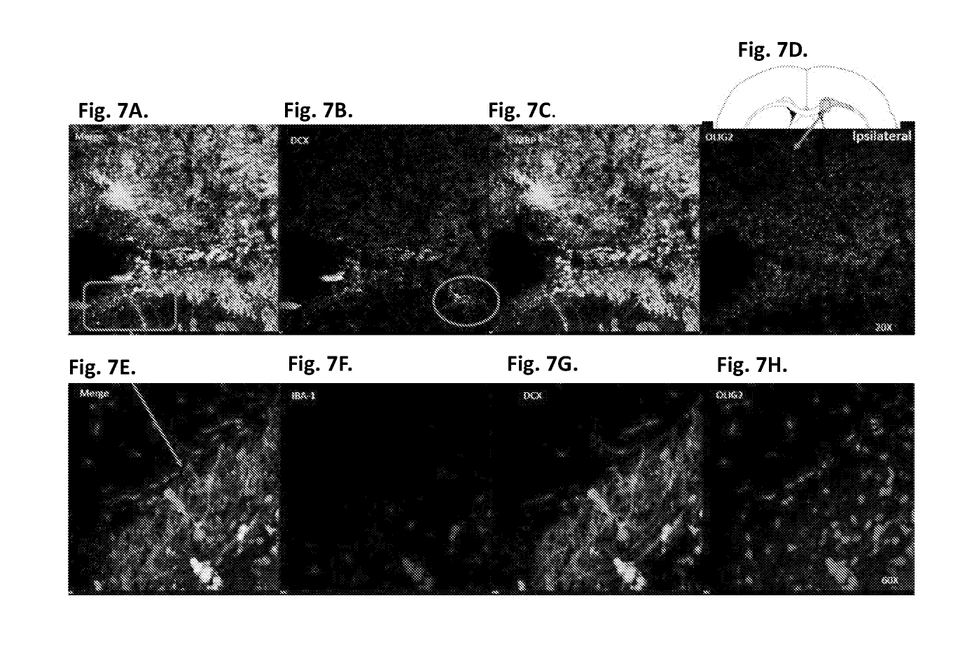

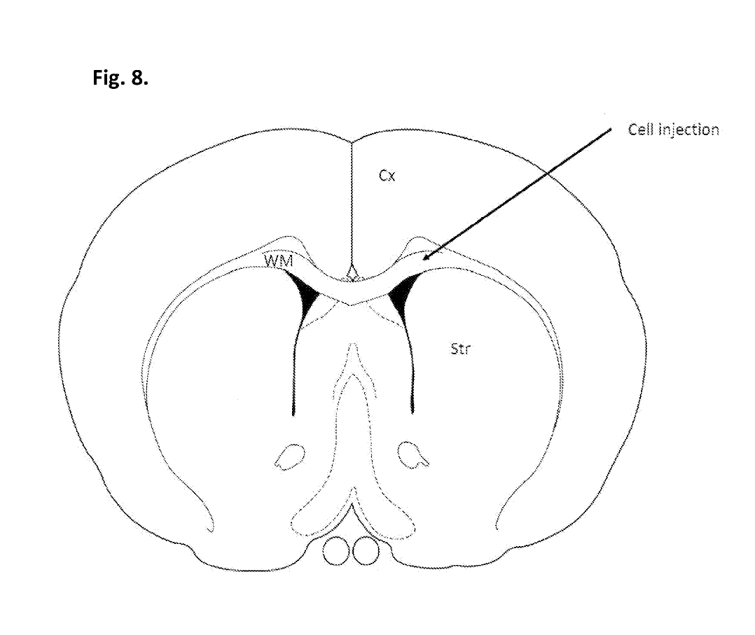

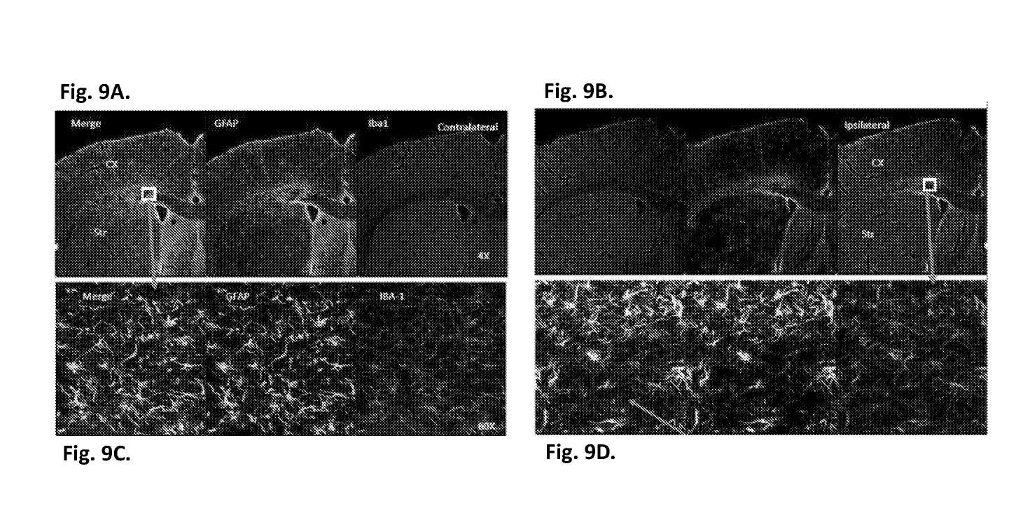

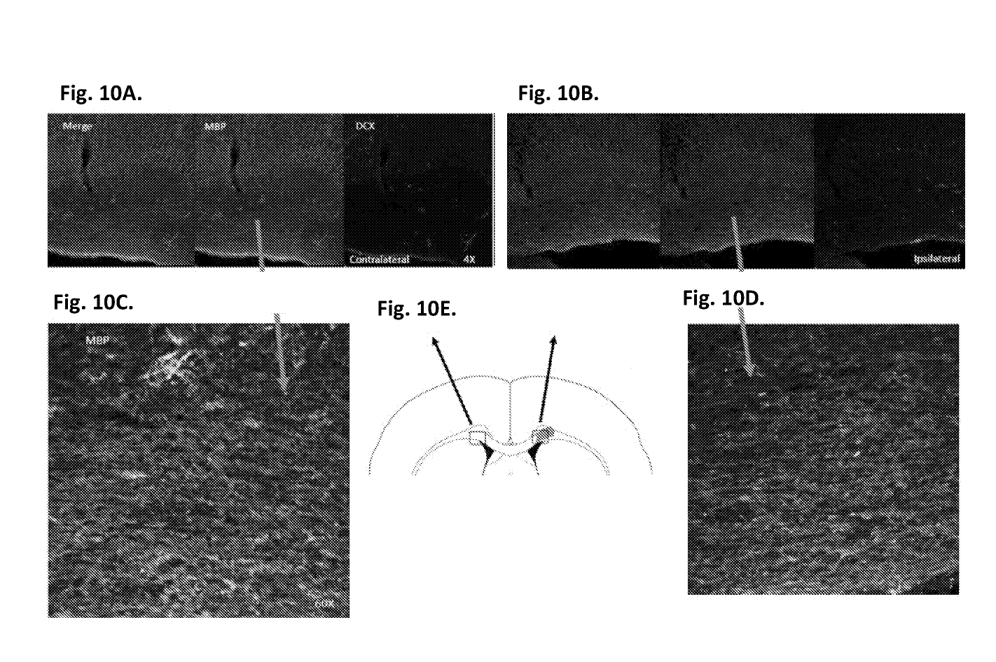

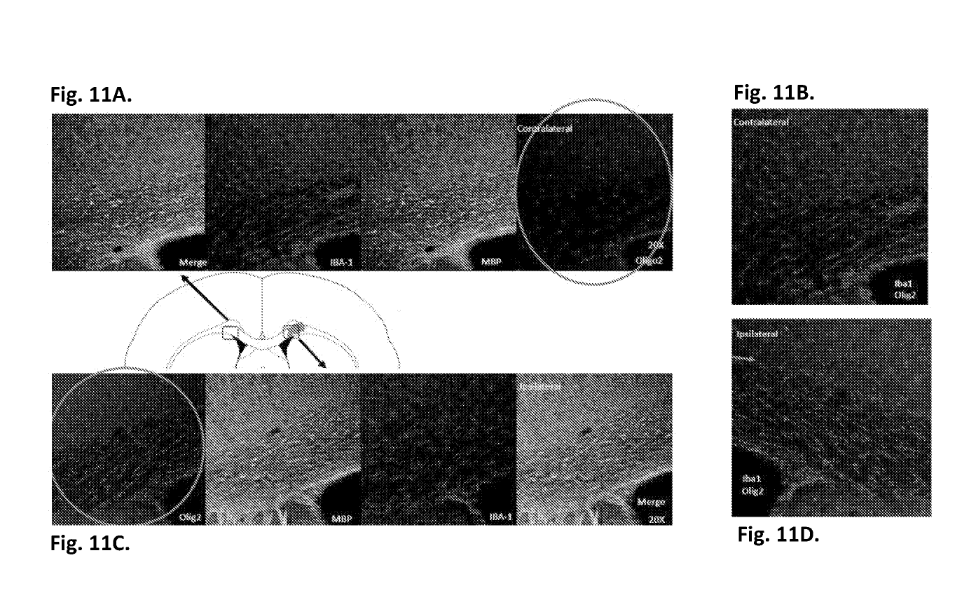

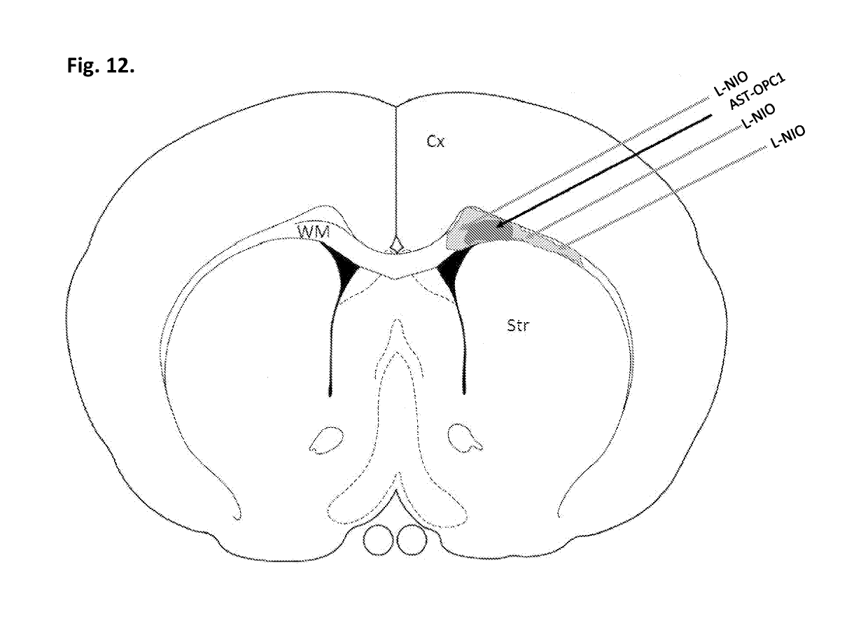

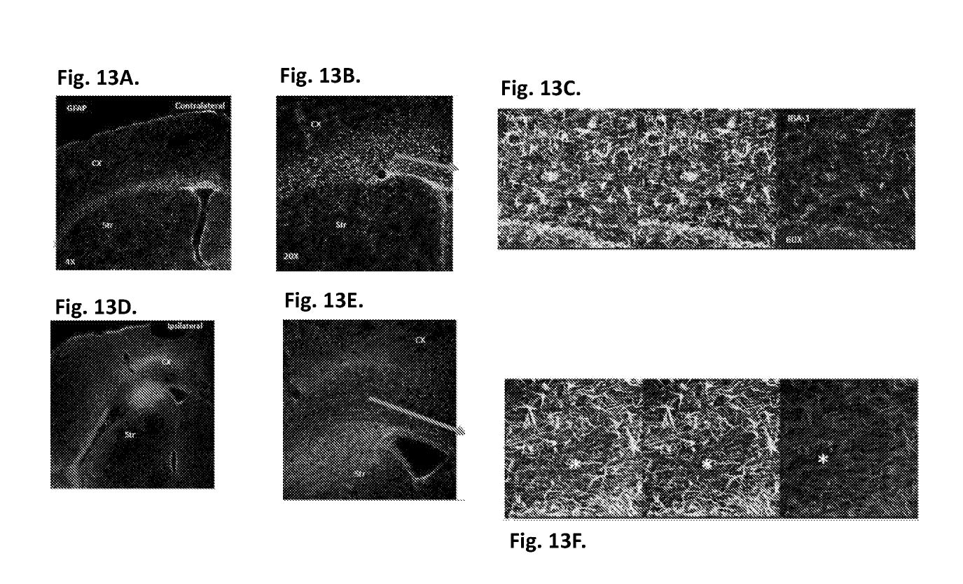

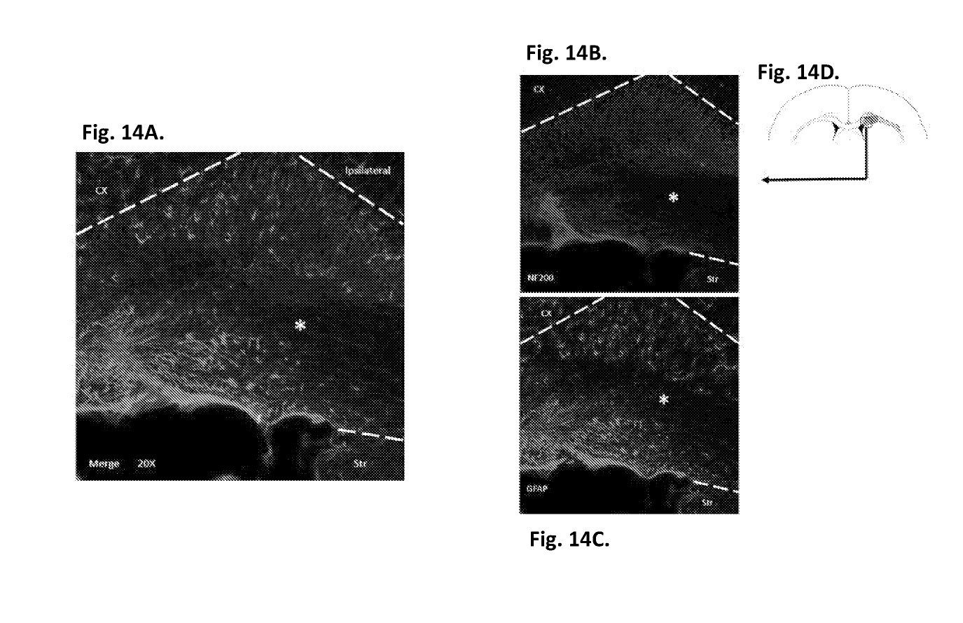

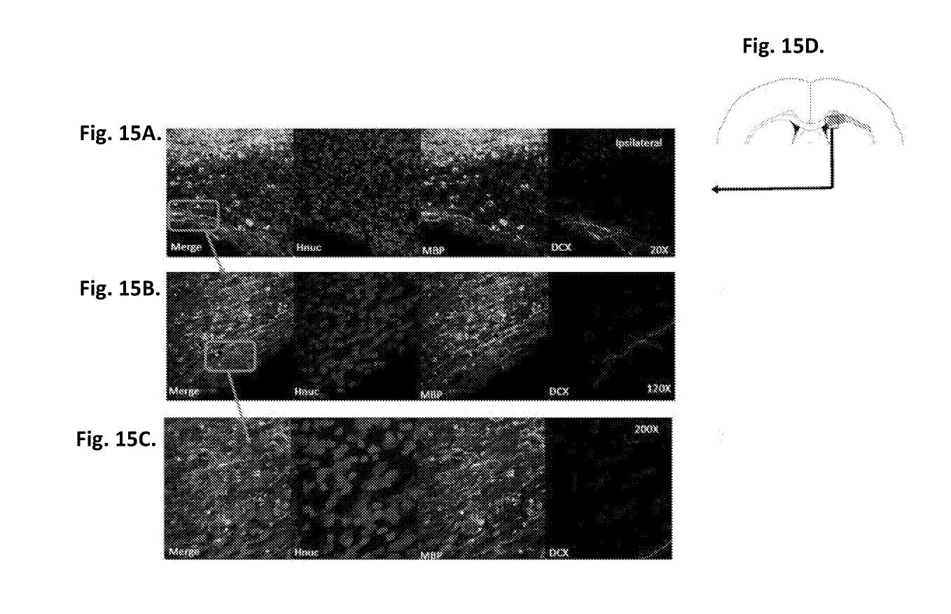

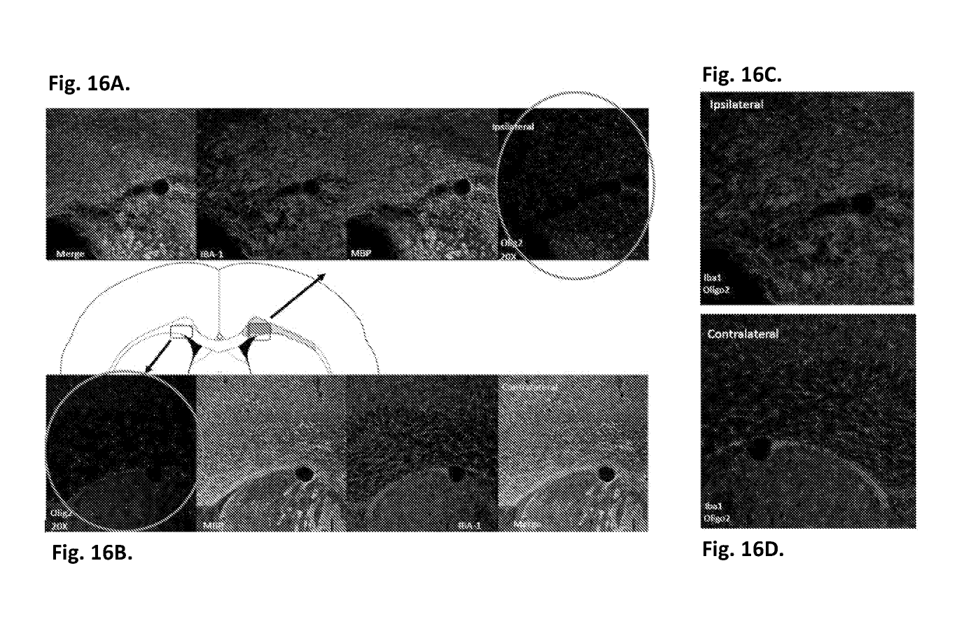

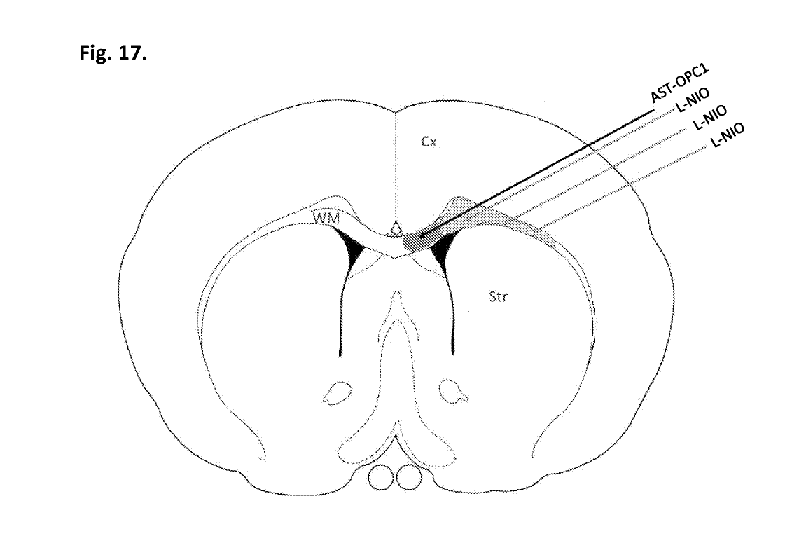

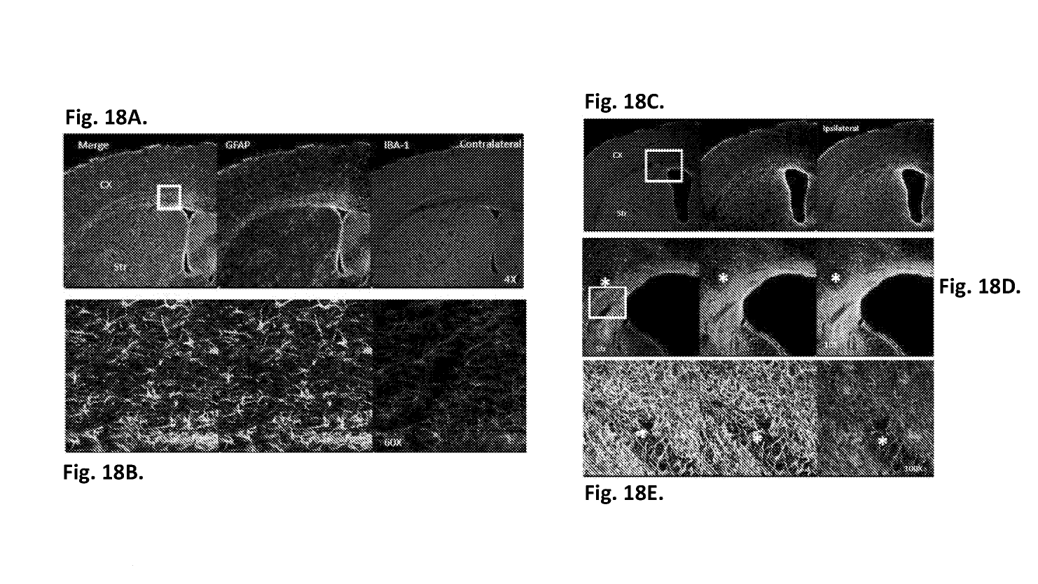

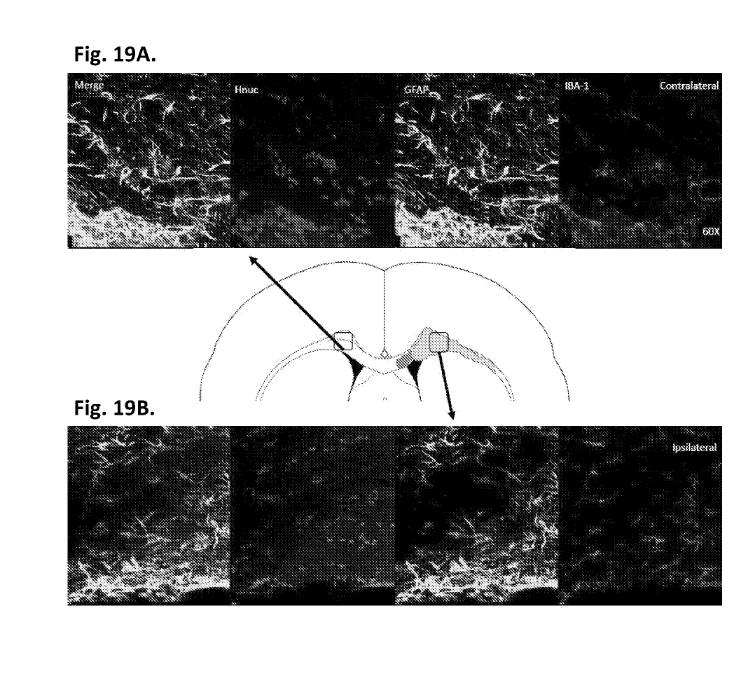

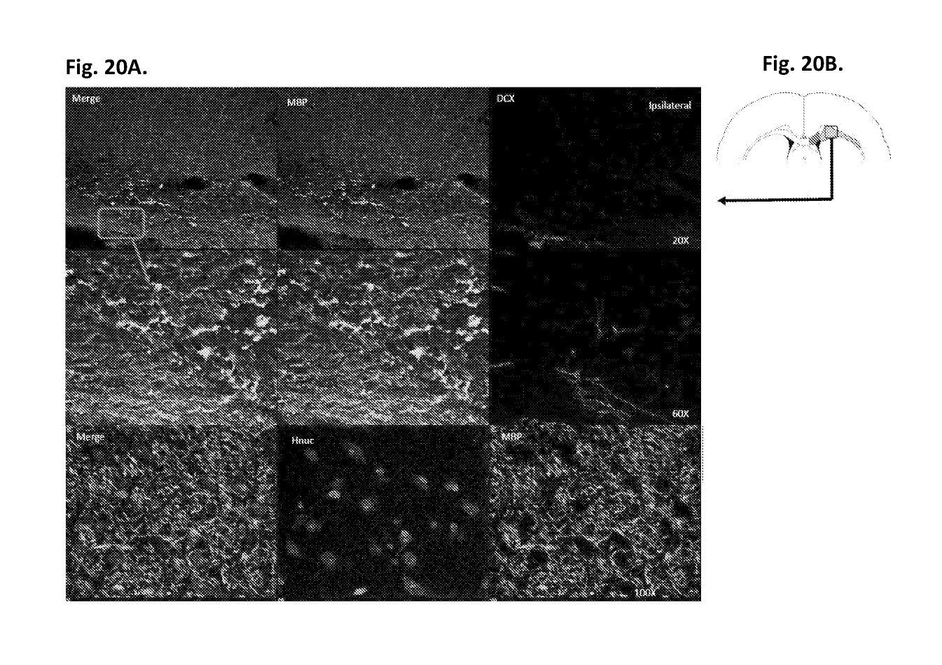

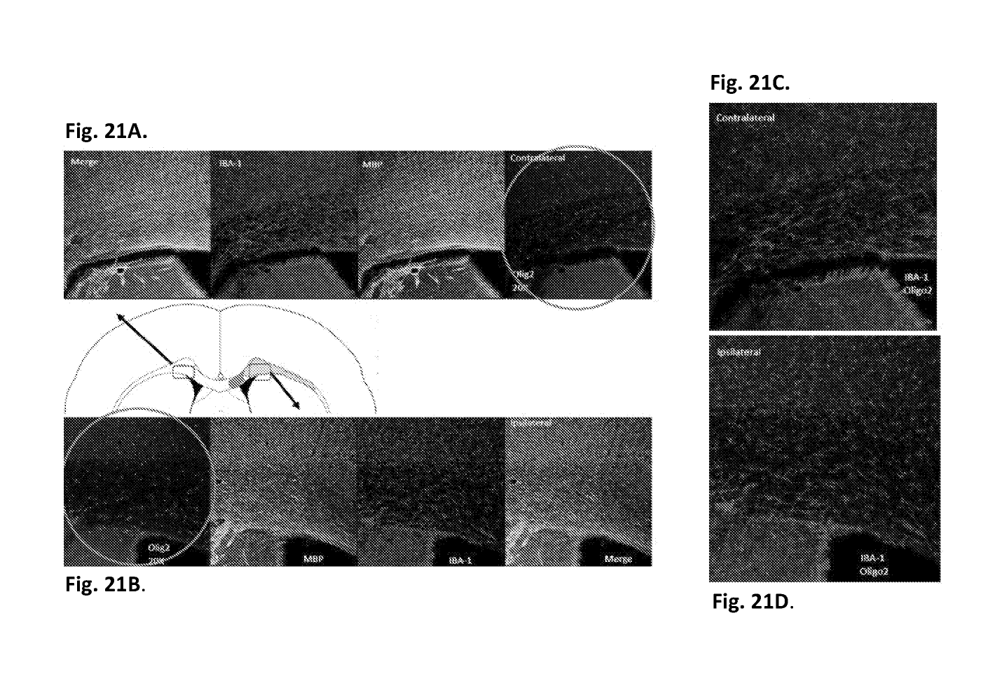

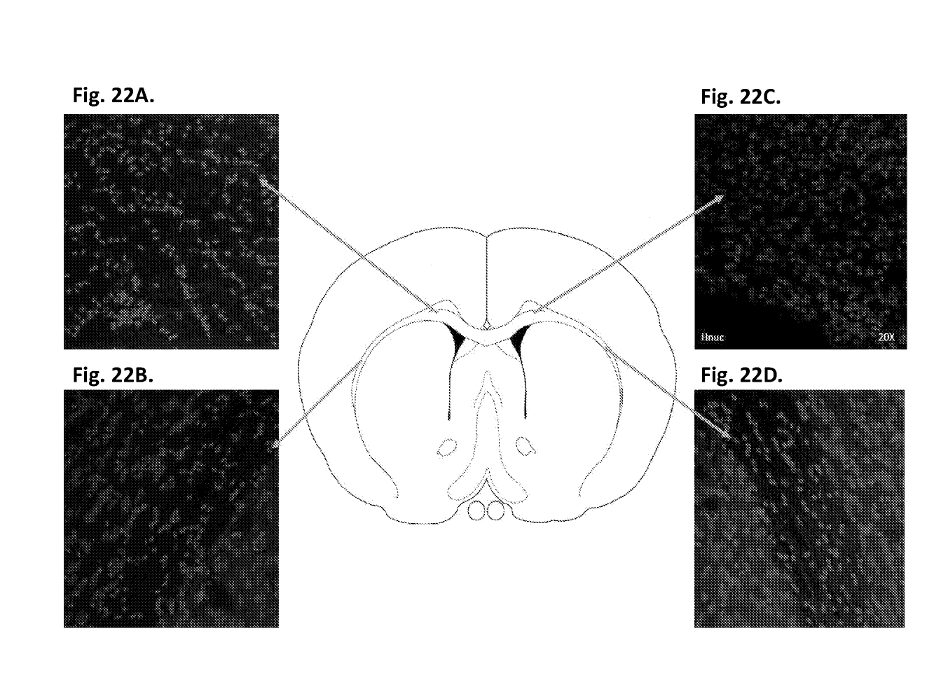

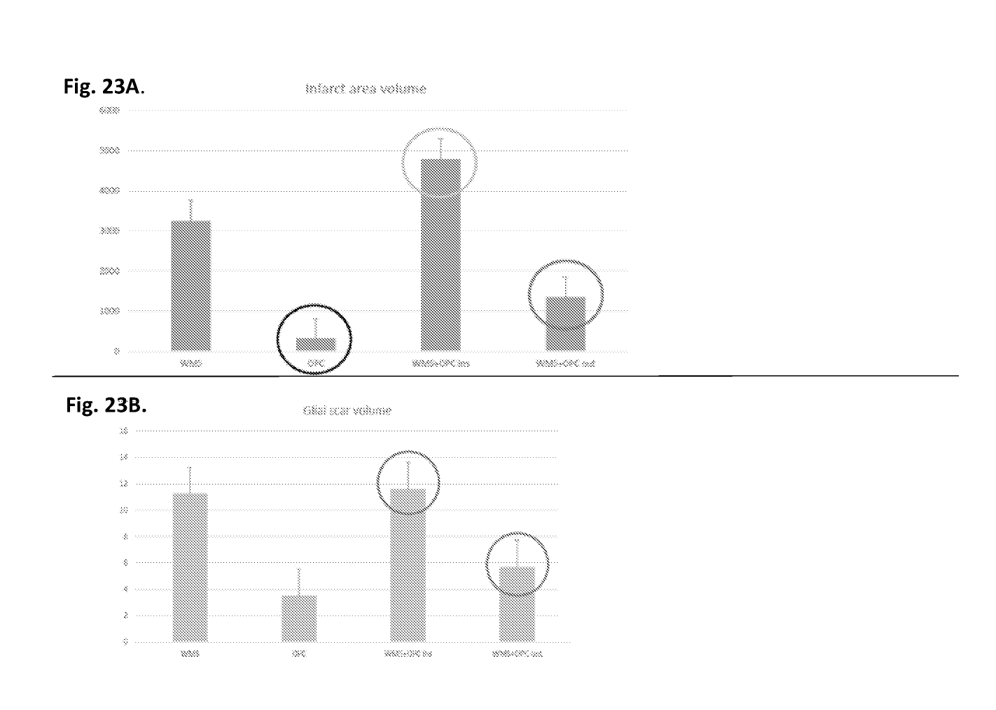

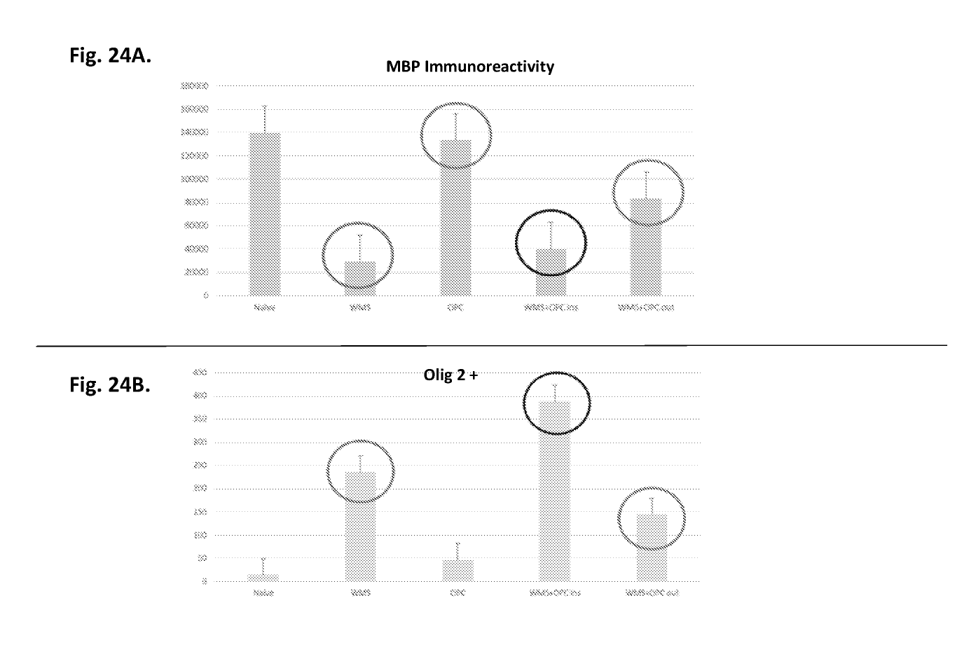

To allow for full study of the AST-OPC1 xenograft transplant, a previously established mouse model of subcortical white matter stroke (Sozmen et al., (2009) J. Neurosci Methods 180(2):261; Hinman et al., (2013) Stroke 44(1):182) that mimics the large white matter lesions seen in moderate to advanced human white matter ischemia or vascular dementia was adapted to the immunodeficient NSG mouse (Shultz et al., (2007) Nat Rev Immunol. 7(20:118; jaxmice.jax.org/nod-scid-gamma). Briefly, to induce focal ischemic lesions, N5-(1-iminoethyl)-L-ornithine, dihydrochloride (L-Nio, Calbiochem), was injected at three stereotactic coordinates directly into the corpus callosum of each mouse brain, as illustrated in FIG. 3. The experimental timeline is illustrated in FIG. 2. The study goals and parameters are described in detail in Table 2. Brain tissue was processed 15 days post stroke induction (i.e. two-weeks post AST-OPC1 or sham injection) and fluorescent immunostaining performed to determine the extent of myelination, axonal loss, astrocyte activation, microglial/macrophage responses and oligodendrocyte responses. Representative results are depicted in FIGS. 1-24.

TABLE 2

| | |
|---|---|
| Goals: | Establish experience in transplantation and |
| | determine early survival and migration |
| | characteristics of AST-OPC1 in NSG white |
| | matter stroke; to understand the effects of the |
| | stroke environment on survival and migration. |
| Groups: | 1) Stroke |
| | 2) Stroke + AST-OPC1 inside infarct |
| | 3) Stroke + AST-OPC1 peri-infarct |
| | 4) Control + AST-OPC1 |
| Sample size: | 5 mice per group |
| AST-OPC1 | 7 days after stroke, 100,000 cells/mouse in a |
| transplantation: | single 1 μL injection delivered inside the |
| | infarct or immediately adjacent to infarct |
| | (peri-infarct) |
| Survival: | 2 weeks post AST-OPC1 or sham injection |

### Example 3: Study 2—Efficacy Study of AST-OPC1 for Behavioral Recovery

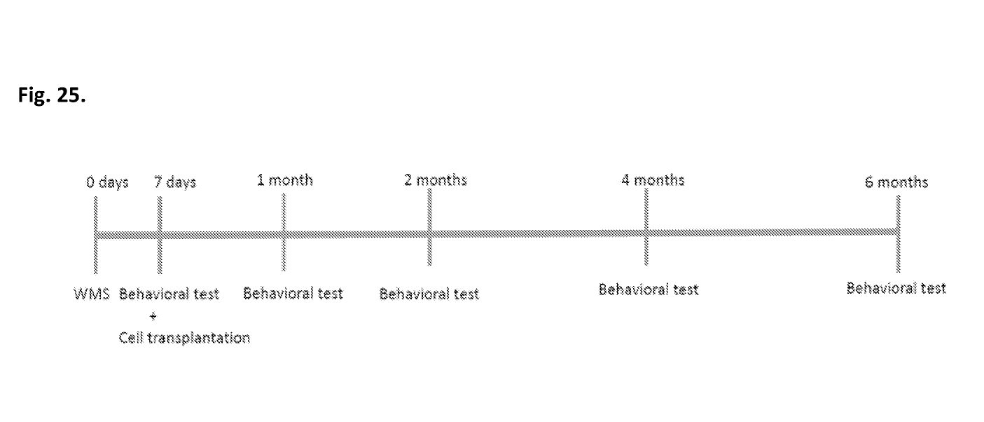

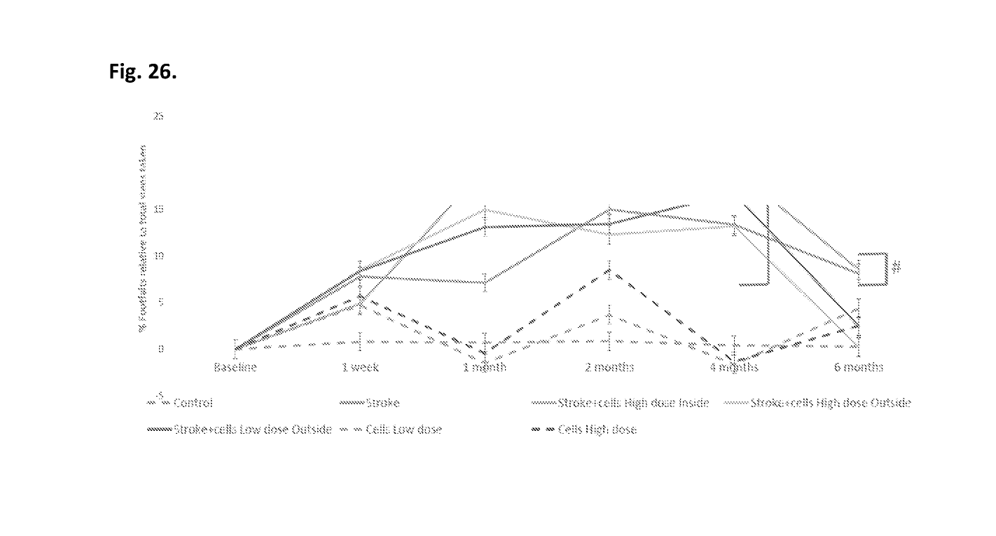

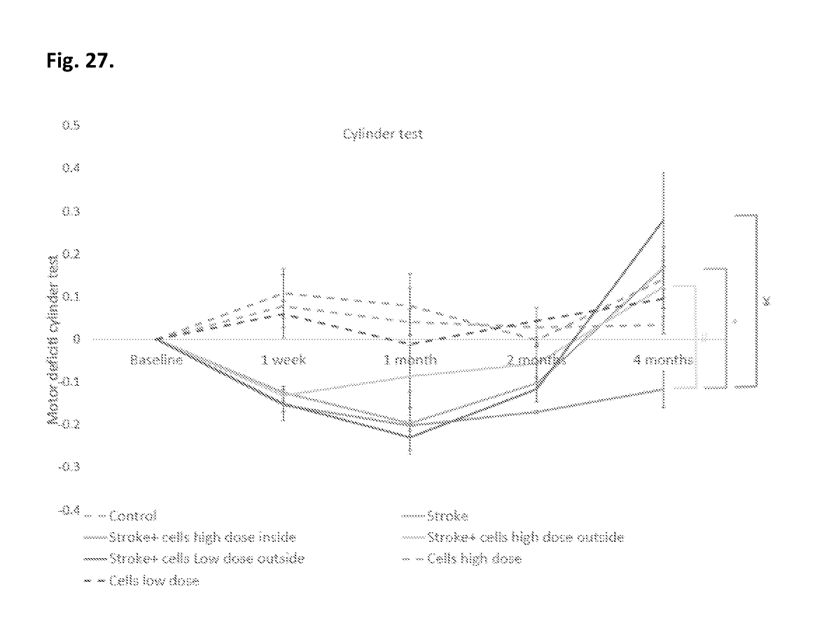

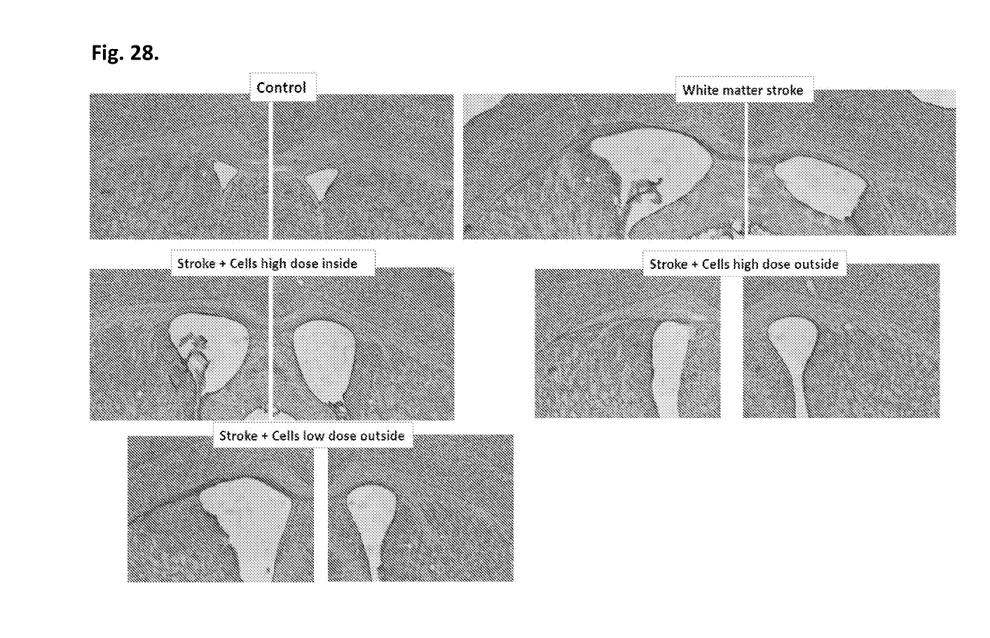

NSG mouse model of WMS as described in Example 2 was used to assess the effect of AST-OPC1 transplantation on behavioral recovery and whether AST-OPC1 transplantation improves white matter preservation based on MRI and ex vivo histochemical staining. The experimental timeline is illustrated in FIG. 25. The study goals and parameters are described in detail in Table 3. The behavioral tests (cylinder test and grid walking) are described in detail in infra. Representative results are depicted in FIGS. 26-28.

TABLE 3

| | |
|---|---|
| Goals: | Determine if OPC1 transplantation at 7 |
| | days after white matter stroke promotes |
| | neurological recovery based on behavior |
| | testing; determine whether OPC1 |
| | transplantation improves white matter |
| | preservation based on MRI and ex vivo |
| | histochemical staining. |
| Groups: | 1) Control (sham surgery) |
| | 2) Stroke alone |
| | 3) AST-OPC1 without stroke (half with |
| | AST-OPC1 low dose, half with AST- |
| | OPC1 high dose) |
| | 4) Stroke + AST-OPC1 low dose peri- |
| | infarct |
| | 5) Stroke + AST-OPC1 high dose peri- |
| | infarct |
| | 6) Stroke + AST-OPC1 high dose inside |
| | infarct |
| Sample Size: | 12 mice per group |
| Animals: | 72 mice total |
| AST-OPC1 transplantation: | 7 days after stroke, 100,000 cells/mouse |
| | (high dose) or 10,000 cells/mouse (low |
| | dose) delivered as a 1 μl injection inside |
| | or immediately outside the infarct (peri- |
| | infarct) |
| Behavior: | Overall goal: To measure proximal and |
| | distal motor control of the impaired |
| | forelimb, as well as hind limb function in |
| | gait. These test natural movements in the |
| | mouse. |
| | Testing time points: pre-stroke (baseline), |
| | 7 days after stroke (before cell |
| | transplantation), monthly from 1-6 months |
| | post-stroke |
| MRI: | Immediately prior to sacrifice and |
| | histological evaluation, a subset of |
| | animals are imaged by MRI to obtain |
| | volumetric measures of intact brain and |
| | white matter |
| Histology: | Upon completion of behavior testing, |
| | brains are processed for histological |
| | evaluations of infarct size, endogenous |
| | brain repair and inflammation, and OPC1 |
| | survival/phenotype |

Cylinder Test.

Exploratory behavior in mice provides a possibility to investigate the neural basis of spatial and motor behavior, which can be used as an assay of brain function. The cylinder test provides a way to evaluate a rodent's spontaneous forelimb use and has been used in a number of motor system injury models of stroke. To evaluate forelimb deficits, the animal is placed in a transparent Plexiglas cylinder and observed. Mice will actively explore vertical surfaces by rearing up on their hind limbs and exploring the surface with their forelimbs and vibrissae. When assessing behavior in the cylinder, the number of independent wall placements observed for the right forelimb, left forelimb and both forelimbs simultaneously are recorded. Animals with unilateral brain damage will display an asymmetry in forelimb use during vertical exploration.

The cylinder task has been found to be objective, easy to use and score, sensitive to chronic deficits that others fail to detect and have high inter-rater reliability.

Grid Walking Test.

The grid walking task, often referred to as the foot fault task, is a relatively simple way to assess motor impairments of limb functioning (most commonly hind limbs, but forelimbs have been evaluated as well) and placing deficits during locomotion in rodents. This task has been found to objectively demonstrate motor coordination deficits and rehabilitation effects after stroke. An animal is placed on an elevated, leveled grid with openings. Animals without brain damage will typically place their paws precisely on the wire frame-to hold themselves while moving along the grid. Each time a paw slips through an open grid, a “foot fault” is recorded. The number of both contra- and ipsilateral faults for each limb is compared to the total number of steps taken and then scored using a foot fault index. Intact animals will generally demonstrate few to no foot faults, and when faults occur, they do so symmetrically. lschemic animals typically make significantly more contralateral foot faults than intact animals. The foot fault test has been shown to be a sensitive indicator for detecting impairments of sensorimotor function after ischemia in rodents.

What is claimed is:

1. A method of improving recovery of a subject after a cerebral ischemic injury comprising administering a therapeutically effective amount of stem cell-derived oligodendrocyte progenitor cells into or directly adjacent to the infarct core in the brain of said subject.

2. The method of claim 1, wherein the cerebral ischemic injury is subcortical white matter stroke.

3. The method according to any one of claims 1-2, wherein the subject is a human.

4. The method according to any one of claims 1-3, wherein the human stem cell-derived oligodendrocyte progenitor cells are administered directly adjacent to the infarct core.

5. The method according to any one of claims 1-3, wherein the human stem cell-derived oligodendrocyte progenitor cells are administered into the infarct core.

6. The method according to any one of claims 1-5, wherein the human stem cell-derived oligodendrocyte progenitor cells are administered during the subacute time period after the ischemic injury.

7. The method according to any one of claims 1-6, wherein the human stem cell-derived oligodendrocyte progenitor cells are administered using a depot delivery system.

8. The method of claim 7, wherein the depot delivery system comprises a hydrogel.

9. The method of claim 8, wherein the hydrogel comprises thiolated hyaluronate.

10. The method according to any one of claims 8-9, wherein the hydrogel comprises thiolated gelatin.

11. The method according to any one of claims 8-10, wherein the hydrogel comprises a crosslinking agent.

12. The method according to any one of claims 1-11, wherein said progenitor cells are derived from adult stem cells.

13. The method according to any one of claims 1-11, where said progenitor cells are derived from induced pluripotent stem cells (IPSCs).

14. The method according to any one of claims 1-11, wherein said progenitor cells are derived from stem cells that are not obtained from embryonic or fetal tissue.

15. The method according to any one of claims 1-14, wherein said cerebral ischemic injury is due to a stroke.

16. The method according to any one of claims 1-14, wherein said cerebral ischemic injury is due to a head injury.

17. The method according to any one of claims 1-14, wherein said cerebral ischemic injury is due to a respiratory failure.

18. A method for improving motor or cognitive function of a subject after a cerebral ischemic injury, said method comprising administering a therapeutically effective amount of stem cell-derived oligodendrocyte progenitor cells into or directly adjacent to the infarct core in the brain of said subject.

19. The method of claim 18, wherein the cerebral ischemic injury is subcortical white matter stroke.

20. The method according to any one of claims 18-19, wherein the subject is a human.

21. The method according to any one of claims 18-20, wherein the stem cell-derived oligodendrocyte progenitor cells are administered directly adjacent to the infarct core.

22. The method according to any one of claims 18-20, wherein the stem cell-derived oligodendrocyte progenitor cells are administered into the infarct core.

23. The method according to any one of claims 18-22, wherein the stem cell-derived oligodendrocyte progenitor cells are administered during the subacute time period after the ischemic injury.

24. The method according to any one of claims 18-23, wherein said progenitor cells are derived from adult stem cells.

25. The method according to any one of claims 18-23, where said progenitor cells are derived from induced pluripotent stem cells (IPSCs).

26. The method according to any one of claims 18-23, wherein said progenitor cells are derived from stem cells that are not obtained from embryonic or fetal tissue.

27. The method according to any one of claims 18-26, wherein said cerebral ischemic injury is due to a stroke.

28. The method according to any one of claims 18-26, wherein said cerebral ischemic injury is due to a head injury.

29. The method according to any one of claims 18-26, wherein said cerebral ischemic injury is due to a respiratory failure.

30. A pharmaceutical composition for the treatment of subcortical white matter stroke, comprising human stem cell-derived oligodendrocyte progenitor cells.

31. The pharmaceutical composition of claim 30, further comprising a depot delivery system.

32. The pharmaceutical composition of claim 31, wherein the depot delivery system comprises a hydrogel.

33. The pharmaceutical composition of claim 32, wherein said hydrogel comprises hyaluronan and/or gelatin.

34. The pharmaceutical composition according to any one of claims 32-33, wherein the hydrogel comprises thiolated hyaluronate.

35. The pharmaceutical composition according to any one of claims 32-34, wherein the hydrogel comprises thiolated gelatin.

36. The pharmaceutical formulation according to any one of claims 32-35, wherein the hydrogel comprises a crosslinking agent.

37. The pharmaceutical formulation according to any one of claims 30-36, wherein said progenitor cells are derived from adult stem cells.

38. The pharmaceutical formulation according to any one of claims 30-37, where said progenitor cells are derived from induced pluripotent stem cells (IPSCs).

39. The pharmaceutical formulation according to any one of claims 30-38, wherein said progenitor cells are derived from stem cells that are not obtained from embryonic or fetal tissue.
